(19) United States
(12) Patent Application Publication
(10) Pub. No.: US 2024/0024514 A1
(43) Pub. Date: Jan. 25, 2024

(54) RECOMBINANT ADENO-ASSOCIATED VIRUSES WITH ENHANCED LIVER TROPISM AND USES THEREOF

(71) Applicants: BEIJING SOLOBIO GENETECHNOLOGY CO., LTD, Beijing (CN); STAIDSON (BEIJING) BIOPHARMACEUTICALS CO. LTD., Beijing (CN)

(72) Inventors: Tingting Zhang, Beijing (CN); Chao Wang, Beijing (CN)

(21) Appl. No.: 18/036,066

(22) PCT Filed: Nov. 16, 2021
(86) PCT No.: PCT/CN2021/130784

§ 371 Date: May 9, 2023

(30) Foreign Application Priority Data
Nov. 16, 2020 (WO) PCT/CN2020/129001

(51) Int. Cl. A61K 48/00; C12N 15/86; C12N 15/113; A61K 38/47; A61P 31/20

(52) U.S. Cl. CPC A61K 48/0066; C12N 15/86; C12N 15/1131; A61K 38/47; A61K 48/0041; A61P 31/20; C12N 2750/14122; C12N 2750/14143; C12N 2310/531

(57) ABSTRACT

Recombinant adenovirus-associated viruses are described. The recombinant adenovirus-associated viruses may include a capsid protein with enhanced tropism for liver cells. The recombinant adenovirus-associated viruses may also exhibit less immunogenicity in humans. The recombinant adenovirus-associated viruses may include expression cassettes including a polynucleotide sequence encoding a therapeutic agent useful in gene therapy treatment of a liver disease. Preparation systems for packaging the recombinant adenovirus-associated viruses, methods of producing the recombinant adenovirus-associated viruses, pharmaceutical compositions including the recombinant adenovirus-associated viruses, and uses of said compositions for treating liver diseases including Fabry disease and Hepatitis B, are also provided.

### SEQUENCE LISTING

The instant application contains a Sequence Listing which has been submitted electronically in ASCII format and is hereby incorporated by reference in its entirety. Said ASCII copy, created on Nov. 6, 2020, is 26 KB in size.

### TECHNICAL FIELD

This disclosure relates generally to recombinant adeno-associated viruses comprising an adeno-associated virus capsid protein with enhanced liver tropism and an expression cassette comprising a polynucleotide sequence encoding a therapeutic agent useful in gene therapy treatment of a liver disease. In some embodiments, the encoded therapeutic agent comprises alpha-galactosidase A (GLA/Gla) or a short hairpin RNA targeting a Hepatitis B virus genome. This disclosure also relates to compositions comprising these recombinant adeno-associated viruses and uses of these recombinant adeno-associated viruses for treating a patient suffering from a liver disease such as Fabry disease or Hepatitis B.

### BACKGROUND

Adeno-associated viruses (AAVs) are small, replication-defective, non-enveloped viruses comprising a single-stranded linear DNA genome. The AAV genome comprises inverted terminal repeats (ITRs) at the ends of the DNA strand, as well as open reading frames (ORFs) encoding replication (Rep) and capsid (Cap) proteins. AAVs have been proven to be generally safe, with no known significant association with the pathogenesis of tumors or other diseases (Guylene (2005) Arch Ophthalmal 123:500-506). In addition to being safe, other characteristics of AAVs render them particularly useful as vectors in gene therapy, such as high infection efficiency, capability for widespread infection, and prolonged expression (David (2007) BMC Bio 7:75). Therefore, AAVs are widely used in the treatment of a plethora of different diseases, such as cancer, retinal diseases, arthritis, acquired immune deficiency syndrome (AIDS), liver diseases, and neurological diseases.

One type of liver disease is Fabry disease, which is a rare genetic disease and belongs to the category of lysosomal storage diseases. It is caused by mutations in the alpha-galactosidase A (gla) gene. The deficiency in GLA results in the accumulation of glycolipids in blood vessels, other tissues, and organs, which may lead to an impairment in their functions. Symptoms include pain, kidney disease, skin lesions, fatigue, nausea, and neuropathy. Treatment includes enzyme replacement therapy using a recombinant GLA. Hepatitis B is a liver disease caused by the Hepatitis B virus (HBV). It is spread when bodily fluids such as blood and semen from an infected person are introduced to an uninfected person. Symptoms include fatigue, poor appetite, stomach pain, nausea, and jaundice. Cirrhosis and liver cancer occur in around 25% of those with chronic Hepatitis B. The World Health Organization (WHO) estimates that around 257 million people in the world were living with chronic Hepatitis B in 2015, with 887,000 deaths resulting from the disease. Therefore, it is important to develop safe, effective methods to treat such widespread liver diseases. AAVs may be used to deliver a therapeutic agent useful in gene therapy treatment of a liver disease such as Fabry disease or Hepatitis B. Treatment of Hepatitis B include gene therapy may involve using a short hairpin RNA (shRNA) targeting a Hepatitis B virus genome. When using AAVs to deliver a therapeutic agent for treating a liver disease, it is desirable that the AAVs have enhanced liver tropism.

AAVs can be classified into many variants, called serotypes, for example AAV1-AAV12 (Gao et al. (2004) J Virol 78: 6381-6388; Mori et al. (2004) Virology 330:375-383; Schmidt et al. (2008) J Virol 82:1399-1406). The hosts of AAVs are usually humans and primates, with AAV1-6 isolated from humans. Therefore, AAV1-6 cause a pronounced immune response in humans. AAV7 and AAV8 were isolated from the heart tissue of rhesus monkeys (Gao et al. (2002) PNAS 99:11854-11859), whereas AAV9-12 were isolated from humans and macaques. While all AAV serotypes share the characteristic icosahedral structure, the variation in both the sequence and surface topology of capsid proteins in different serotypes of AAV gives them differential cell surface receptor binding ability and tropism for different cell types (Timpe (2005) Curr Gene Ther 5:273-284). For example, AAV2 has tropism for a variety of cell types, with particularly pronounced tropism for neurons; AAV1 and AAV7 have enhanced tropism for skeletal muscle; AAV3 has enhanced tropism for megakaryocytes; AAV5 and AAV6 possess enhanced tropism for airway epithelial cells; whereas AAV8 has the best-known tropism for liver cells.

Natural AAVs have limited tropism profiles and the efficacy of therapeutics comprising different AAV capsid protein for their respective target cells varies greatly. Furthermore, humans and other primates often produce neutralizing antibodies against natural AAV variants, resulting in a decreased half-life of AAV and loss of efficacy after administration. Thus, there has been prolific research on genetically engineering AAV capsids with increased tropism for specific cell types and reduced immunogenicity. Engineered AAVs have already been utilized in the clinic. For example, AAV2.5 comprises a chimeric capsid made from adding five amino acids responsible for skeletal muscle tropism from AAV1 capsid to the capsid of AAV2. AAV2.5 comprising the minidystrophin gene has been used to treat Duchenne muscular dystrophy (Bowles et al. (2012) Mol Ther 20:443-455), with phase I clinical trials already completed. The safety of these engineered AAVs has also been evaluated. It was shown that AAV2.5 not only has enhanced tropism for skeletal muscle, but also is less immunogenic compared to natural AAV2.

There remains a need for genetically engineered AAVs that exhibit enhanced liver tropism and decreased immunogenicity in humans. Such AAVs provide a valuable and improved tool for delivering a therapeutic agent for treating a liver disease such as Fabry disease or Hepatitis B.

### SUMMARY

Some aspects of this disclosure relate to recombinant AAV (rAAV). In some embodiments, the rAAV possesses improved properties such as high packaging yield, increased levels of gene expression, lower immunogenicity, and/or enhanced tropism for liver cells. In these embodiments, the rAAV comprises an expression cassette comprising a polynucleotide sequence encoding a therapeutic agent for the treatment of a liver disease. In some embodiments, the therapeutic agent is an shRNA or GLA. In certain embodiments, the liver disease is Hepatitis B or Fabry disease. Aspects of the disclosure further relate to a vector containing shRNA or GLA expression cassettes, such as a plasmid comprising the expression cassette. Aspects of the disclosure further relate to a packaging system for producing the rAAV of the invention. Aspects of the disclosure further relate to a plasmids system for packaging the rAAV of the invention. Aspects of the disclosure also relate to a cell comprising a plasmid system for packaging the rAAV of the invention, including an isolated engineered cell comprising the packaged rAAV. Aspects of the disclosure also relate to a method of packaging the rAAV of the invention. Aspects of the disclosure further relate to a composition comprising the rAAV. Aspects of the disclosure also relate to the use of a composition comprising the rAAV of the invention in the preparation of a medicament for prevention and treatment of liver diseases such as Hepatitis B and Fabry disease, and its use in a method of treating liver diseases such as Hepatitis B and Fabry disease.

Additional features and advantages of the disclosed embodiments are set forth in part in the description that follows, and in part will be evident from the description, or may be learned by practice of the disclosed embodiments. The features and advantages of the disclosed embodiments will be realized and attained by the elements and combinations particularly pointed out in the accompanying claims.

Both the foregoing general description and the following detailed description are mere examples and explanatory, which are not restrictive of the disclosed embodiments as claimed.

The accompanying drawings constitute a part of this specification. The drawings illustrate several embodiments of the present disclosure and, together with the description, serve to explain the principles of the disclosed embodiments as set forth in the accompanying claims.

### BRIEF DESCRIPTION OF FIGURES

The foregoing and other objects, features, and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure.

FIGS. 1A-1I illustrate maps of various plasmids. Specifically, FIGS. 1A-1D illustrate maps of various plasmids which contain different promoters and a nucleotide sequence encoding Gluc. FIGS. 1E-1I illustrate maps of various plasmids which contain different promoters and a nucleotide sequence encoding GLA.

FIGS. 2A-2B illustrate maps of plasmids comprising a nucleotide sequence encoding an shRNA targeting an HBV genome.

FIGS. 3A-3F are graphs showing the tropism of AAV2/8, AAV2/3B, AAV2/7, and AAV2/9 for various liver cell lines.

FIGS. 4A-4J are graphs showing the tropism of AAV2/8 and AAV2/X for various liver cell lines.

FIGS. 5A-5J are graphs showing the tropism of AAV2/8 and AAV2/X for human primary liver cancer cells.

FIGS. 6A-6N show fluorescent images of in vivo EGFP expression in different tissues of macaques administered with the AAV2/X-CMV-EGFP vector, including in heart (FIG. 6A), lung (FIG. 6B), liver (FIGS. 6C-6G), brain (FIG. 6H), testicle (FIG. 6I), biceps femoris (FIG. 6J), stomach (FIG. 6K), jejunum (FIG. 6L), kidney (FIG. 6M), and spleen (FIG. 6N), respectively.

FIGS. 7A-7N show fluorescent images of in vivo EGFP expression in different tissues of macaques administered with the AAV2/8-CMV-EGFP vector, including in heart (FIG. 7A), lung (FIG. 7B), liver (FIGS. 7C-7G), brain (FIG. 7H), testicle (FIG. 7I), biceps femoris (FIG. 7J), stomach (FIG. 7K), jejunum (FIG. 7L), kidney (FIG. 7M), and spleen (FIG. 7N), respectively.

FIG. 8 is a chart showing comparative levels of Nabs against AAV2/8 or AAV2/X in pooled human serum.

FIGS. 9A-9B show schematics of expression cassettes comprising different promoters and polynucleotides encoding different transgenes.

FIGS. 10A-10B are graphs showing expression levels of Gluc and GLA under different promoters. FIG. 10A shows the expression level of Gluc under DC172 promoter, DC190 promoter or CMV promoter. FIG. 10B shows the expression levels of GLA under DC172 promoter or LP1 promoter.

FIG. 11 shows a schematic of an expression cassette containing the WPRE sequence.

FIGS. 12A-12B are graphs showing comparison of GLA activity using AAV2/X containing the DC172 promoter or the LP1 promoter with and without the addition of WPRE sequence in normal mice.

FIGS. 13A-13D are graphs showing the activity of GLA in different organs of model mice administered with AAV2/X or AAV2/8.

FIGS. 14A-14D are graphs showing the activity of GLA in different organs of model mice administered with AAV2/X of different MOI.

FIGS. 15A-15B are graphs showing the Hepatitis B surface antigen (HBsAg) levels reduced by shRNA encoded by a nucleotide sequence which was contained in the AAV2/X or AAV2/8.

FIGS. 16A-16B are graphs showing the Hepatitis B e-antigen (HBeAg) levels reduced by shRNA encoded by a nucleotide sequence which was contained in the AAV2/X or AAV2/8.

FIGS. 17A-17B are graphs showing the levels of HBV DNA reduced by shRNA encoded by a nucleotide sequence which was contained in the AAV2/X or AAV2/8.

### DETAILED DESCRIPTION

While examples and features of disclosed principles are described herein, modifications, adaptations, and other implementations are possible without departing from the spirit and scope of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items, or meant to be limited to only the listed item or items. It should also be noted that as used in the present disclosure and in the appended claims, the singular forms “a,” “an,” and “the” include plural references unless the context clearly dictates otherwise. Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. Except as otherwise indicated, standard methods known to those skilled in the art may be used for the production of recombinant and synthetic polypeptides, manipulation of nucleic acid sequences, production of transformed cells, construction of rAAV constructs, modified capsid proteins, and vectors expressing the AAV Rep and/or Cap proteins, and transiently and stably transfected packaging cells.

### Recombinant AAVs

Some aspects of this disclosure relate to a recombinant AAV (rAAV). As used herein, the term “recombinant AAV” generally refers to an infectious, replication-defective AAV virus modified to provide particular properties and/or comprising therapeutic nucleic acids of interest. In some embodiments, the virus may comprise a wild-type AAV capsid and a genome that has been modified. In some embodiments, the virus may comprise a modified AAV capsid. The term “wild-type AAV genome” refers to an unmodified, linear, ssDNA molecule comprising ITRs at both ends. The ITRs provide origins of replication for the viral genome. The wild-type AAV genome may also comprise ORFs encoding the capsid (Cap) and replication (Rep) proteins. The Cap protein is a structural protein which forms a shell enclosing the AAV genome. The rep proteins are non-structural proteins which play a role in AAV replication and packaging.

The Cap protein is encoded by the cap genes. The capsid protein of an AAV may play an important role in determining the tropism of the virus. As used herein, the term “tropism” generally refers to the preferential entry of the virus into certain cell or tissue type(s) and/or preferential interaction with the cell surface that facilitates entry into certain cell or tissue types. As used herein, the term “tropism profile” refers to the pattern of viral transduction of one or more target cells, tissues and/or organs. For example, some AAV capsids may exhibit efficient transduction of neurons, but not heart tissue. The tropism profile of an AAV may be altered via modification of the capsid protein. Various methods of modifying AAV capsids are known in the art. For example, in U.S. Pat. No. 9,186,419, AAV capsid proteins with modified tropism profiles were produced via scrambling or shuffling of two or more different AAV capsid sequences that combine portions of two or more capsid protein sequences. A rAAV containing a scrambled capsid is referred to as a chimeric or mosaic AAV.

In some embodiments, the rAAVs of this disclosure are mosaic AAVs comprising a capsid protein which displays enhanced tropism for liver cells of an organism compared to corresponding AAVs of other serotypes. In some embodiments, the organism is a mammal. In a particular embodiment, the organism is a human. As will be appreciated by one skilled in the art, AAV8 was the serotype with the best-known tropism for liver cells. In some embodiments, the rAAV described herein comprises a capsid protein with superior tropism for liver cells compared to a corresponding rAAV comprising a capsid protein of the AAV8. In one embodiment, the rAAV is a mosaic AAV which comprises a capsid protein (AAVX) with the amino acid sequence of SEQ ID NO:1. In a particular embodiment, the rAAV comprises a capsid protein with the amino acid sequence of SEQ ID NO: 1 (AAVX) and at least one ITR from AAV2 serotype, which is denoted AAV2/X.

AAVs have been known to elicit immune responses in humans. Consistent with disclosed embodiments, a rAAV comprising a mosaic capsid may exhibit reduced immunogenicity in an organism compared to a corresponding rAAV comprising a capsid protein from a different serotype. In some embodiments, the organism is a mammal. In a particular embodiment, the organism is a human. The term “immunogenicity” as used herein generally refers to the strength of the immune response elicited by an antigen. The term “immune response” generally refers to the process by which an organism recognizes and defends itself against bacteria, viruses, and other substances, both living and nonliving, that appear foreign and harmful. Such substances are commonly referred to as “antigens.” Two types of immune responses are found in humans: innate immune response and acquired immune response. Innate immune responses are not specific to a particular antigen, whereas acquired immune responses develop after exposure to an antigen. Host immune responses during administration of rAAVs may negatively impact long-term transgene expression in humans, reduce efficacy of the delivered therapeutic agent, and/or elicit undesired side effects. It is estimated that over 90% of the human population has been exposed to wild-type AAVs, which may lead to the development of pre-existing immune responses that can inhibit the clinical efficacy of certain serotypes of administered rAAV. For example, about 70% of the population in the world has been circulating neutralizing antibodies (NAb) against AAV1 and AAV2. As will be understood by one skilled in the art, the term “neutralizing antibody” generally refers to an antibody that is part of the adaptive immune response which defends a cell from a pathogen or infectious particle by specifically binding to surface structures on an infectious particle, thereby rendering it unable to interact with its host cells. In some embodiments, the rAAVs described herein exhibit reduced immunogenicity compared to corresponding rAAVs comprising capsid protein from other serotypes. In a particular embodiment, the rAAVs of this disclosure exhibit reduced immunogenicity compared to a corresponding rAAV comprising a capsid protein from AAV8. In some embodiments, the rAAV comprise a capsid protein of the amino acid sequence of SEQ ID NO: 1. In some embodiments, the capsid protein with the amino acid sequence of SEQ ID NO: 1 is encoded by a polynucleotide sequence at least 70%, 75%, 80%, 85%, 90%, 95%, 96%, 97%, 98%, or 99% identical to SEQ ID NO: 10. In some embodiments, the capsid protein of the amino acid sequence of SEQ ID NO: 1 is encoded by the polynucleotide sequence of SEQ ID NO: 10. In a particular embodiment, the rAAV is the AAV2/X (comprising a capsid of SEQ ID NO: 1 and ITRs from AAV2).

Aspects of this disclosure include a rAAV comprising an expression cassette. In some embodiments, the expression cassette may include at least a portion of a wild-type AAV genome. In certain embodiments, the expression cassette may comprise at least one polynucleotide sequence encoding a therapeutic agent. The term “expression cassette” generally refers to a nucleic acid sequence comprising at least one polynucleotide sequence encoding a therapeutic agent, components necessary for the expression of the therapeutic agent, and other nucleic acid sequences. The therapeutic agent may be utilized for the treatment of a condition or a disease, including a liver disease. Non-limiting exemplary liver diseases include Fabry disease, Hepatitis B, Hemophilia A, Hemophilia B, Crigler Najjar, Wilson disease, OTC deficiency (omithine transcarbamylase deficiency), glycogen storage disease type I a (GSD I a), Citrullinemia type I, Methylmalonic acidemia and other diseases. In a particular embodiment, the liver disease is Hepatitis B. In another embodiment, the liver disease is Fabry disease.

By way of example, the therapeutic agent may comprise a polypeptide, a peptide, or a nucleic acid. In some embodiments, the therapeutic agent is an antibody or an antigen-binding fragment thereof, a therapeutic peptide, or an shRNA. In some embodiments, the therapeutic agent is an shRNA targeting an HBV genome. In some embodiments, the shRNA has the sequence of SEQ ID NO: 3. In an alternate embodiment, the therapeutic agent is GLA useful for treating Fabry disease. In some embodiments, the GLA has the amino acid sequence of SEQ ID NO: 2.

RNA interference (RNAi) is a ubiquitous biological process found in organisms ranging from animals to plants and fungi. RNAi controls gene expression in an organism via the regulation of messenger RNA (mRNA). During this process, double-stranded RNA (dsRNA) is cut into small fragments of around 20 to 25 nucleotides in length by an enzyme called Dicer. These fragments, referred to as small interfering RNA (siRNA), sometimes called short interfering RNA or silencing RNA, are double-stranded and may be loaded onto an RNA-induced silencing complex (RISC), a protein complex which includes Argonautes family proteins. Upon binding, one strand of the dsRNA is removed, allowing the remaining strand to become available to bind to mRNA sequences via standard Watson-Crick base pairing. This binding results in either the cleavage and destruction of the target mRNA by the Argonaute protein, or recruitment of other factors that regulate the mRNA. A short hairpin RNA (shRNA) is an artificial RNA molecule which forms a hairpin structure comprising a stem region of paired sense and antisense strands, connected by unpaired nucleotides which form a loop. ShRNA also includes two short inverted repeat sequences. After shRNA enters the cell, it is unwound by RNA helicase in the host cell into a sense RNA strand and an antisense RNA strand. The antisense RNA strand binds to RISC, recognizes and interacts with the mRNA containing a sequence complementary to the antisense strand. Subsequent cleavage and degradation of the mRNA by RISC results in downregulation of the target gene. ShRNA is used widely in research and in the clinic, due to features such as gene sequence specificity, efficiency, and hereditability. Various methods of introducing shRNA into cells are commonly used, including direct plasmid delivery, and through viral or bacterial vectors. The method of plasmid or viral vector-mediated expression of shRNA in vivo exhibits advantages over direct synthesis of siRNA. The dsRNA sequence corresponding to the shRNA is cloned into a plasmid vector or a viral vector containing a suitable promoter, after which the cell is transfected with the plasmid or infected with the virus, and the desired shRNA transcribed under the control of the promoter. AAV is a commonly used virus for shRNA delivery. Although AAVs are often used as vectors for delivering shRNAs into cells, various aspects of shRNA delivery using AAV vectors still need to be optimized. For example, there is a need for AAV exhibiting superior tropism for liver cells and/or low immunogenicity in humans. Additionally, the short length of shRNA sequences often results in low packaging yield in vitro and transgene expression in the host organism. Therefore, there is a need to optimize packaging efficiency and transgene expression levels.

According to some embodiments, the expression cassette may also include at least one filler sequence. As used herein, the term “filler sequence” generally refers to a nucleic acid sequence other than the at least one polynucleotide encoding a therapeutic agent and components necessary for the transcription and expression of said therapeutic agent. In some embodiments, the filler sequence is selected to be of a length such that the length of the expression cassette is proximate to the length of a wild-type AAV genome. As used herein, the term “proximate” is of equivalent meaning to the term “substantially similar”. In some embodiments, the length of the expression cassette is between about 3.2 kb to about 5.2 kb in length. In alternate embodiments, the length of the expression cassette is between about 1.6 kb to about 2.6 kb in length. In certain embodiments, the length of the expression cassette may be more than 5.2 kb or less than about 1.6 kb in length, depending on the properties of the polynucleotide sequence encoding the therapeutic agent or the application of the rAAV. By way of example, the filler sequence may comprise a non-encoding sequence. The term “non-encoding” generally refers to a nucleic acid sequence which does not encode a protein or other biologically active molecule. For example, the non-encoding sequence may be an intron or a gene regulatory element. In particular embodiments, the non-encoding sequence is a human non-encoding sequence. Optionally, the human non-encoding sequence is inert or innocuous, that is, it does not have function or activity. Non-limiting exemplary human non-encoding sequences include a fragment, or combination of a plurality of sequence fragments, of an intron sequence of human factor IX, a sequence of human cosmid C346, or an HPRT-intron sequence. For example, the filler sequence may comprise HPRT-intron 2 sequence of SEQ ID NO: 4. The filler sequence may be located upstream or downstream of the at least one polynucleotide encoding the therapeutic agent. In a preferred embodiment, the at least one polynucleotide encoding the therapeutic agent is located upstream of the at least one filler sequence. For example, the at least one polynucleotide encoding the therapeutic agent may encode an shRNA and the at least one filler sequence located downstream of the shRNA.

Consistent with embodiments of this disclosure, the expression cassette may also comprise a promoter located upstream of the at least one polynucleotide sequence encoding a therapeutic agent and at least one filler sequence, if present. As will be appreciated by one skilled in the art, the promoter may be any type of promoter, depending on the application for which the rAAV is utilized, including constitutive and inducible promoters. In some embodiments, the promoter is an RNA polymerase II promoter or an RNA polymerase III promoter. Exemplary promoters include, without limitation, an LP1 promoter, an ApoE/hAAT promoter, a DC172 promoter, a DC190 promoter, an ApoA-I promoter, a TBG promoter, an LSP1 promoter, a 7SK promoter, an H1 promoter, a U6 promoter, and an HDIFN promoter. In a particular embodiment, the promoter is an H1 promoter comprising the sequence of SEQ ID NO: 9.

In accordance with embodiments of this disclosure, the expression cassette may also comprise at least one AAV ITR. As will be appreciated by one skilled in the art, the term “ITR” refers to sequences of about 145 nucleotides in length, which are derived from the termini of a wild-type AAV genome. The ITR sequence may be required for replication and packaging of the AAV. The at least one ITR of the rAAV disclosed herein may be from any serotype of AAV, including clades A-F, AAV1, AAV2, AAV3, AAV4, AAV5, AAV6, AAV7, AAV8, AAV9, or any hybrid/chimeric types thereof. In a particular embodiment, the ITR is from AAV2.

In some disclosed embodiments, the rAAV comprises an expression cassette which is single-stranded. In alternative embodiments, the rAAV comprises an expression cassette which is double-stranded, or self-complementary (scAAV). scAAV vector genomes contain DNA strands which intramolecularly anneal to form double-stranded DNA following uncoating in target cells. Due to skipping of second strand synthesis, scAAVs allow for rapid expression in the target cell. In some embodiments, the rAAV comprises a single-stranded expression cassette that is between about 3.2 kb to about 5.2 kb in length. In alternate embodiments, the rAAV comprises a double-stranded expression cassette that is between about 1.6 kb to about 2.6 kb in length. In some embodiments of this disclosure, the expression cassette further comprises at least one reporter sequence located downstream of the promoter sequence. Examples of reporters include Gaussia luciferase, and fluorescent proteins, such as EGFP.

Some aspects of this disclosure relate to a rAAV which exhibit increased therapeutic agent expression in the host cell or organism compared to a corresponding rAAV of another serotype. Alternatively, or additionally, the rAAV disclosed herein exhibits greater packaging yield compared to a corresponding rAAV of another serotype. In some embodiments, the corresponding rAAV is of the AAV8 serotype. Methods of measuring gene expression are known in the art. Exemplary methods include, but are not limited to, quantitative polymerase chain reaction (qPCR), Western blot, Northern blot, and fluorescence microscopy using a reporter gene.

In an embodiment, the rAAV comprises a capsid protein comprising the amino acid of SEQ ID NO: 1, an expression cassette comprising two ITRs from AAV2 serotype, and from 5′ to 3′, a promoter, a polynucleotide encoding an shRNA, and a human non-encoding filler sequence. In some embodiments, the shRNA targets the genome of the Hepatitis B virus (HBV). In certain embodiments, the polynucleotide encoding an shRNA comprises the sequence SEQ ID NO: 3. In some embodiments, the filler sequence comprises the sequence of SEQ ID NO: 4. In particular embodiments, the promoter comprises the sequence of SEQ ID NO: 9. In some embodiments, the expression cassette comprises the sequence of SEQ ID NO: 5. In another particular embodiment, the rAAV comprises a capsid protein comprising the amino acid sequence of SEQ ID NO: 1, an expression cassette comprising two ITRs from AAV2 serotype, and from 5′ to 3′, a promoter and GLA. In another embodiment, the amino acid sequence of the GLA comprises SEQ ID NO: 2. In one embodiment, the expression cassette comprises the sequence of SEQ ID NOs: 6-7.

### Virus Packaging

Aspects of this disclosure include rAAV plasmids system, cells used for packaging of the rAAVs disclosed herein, and methods for packaging the rAAVs disclosed herein. As used herein, the terms “virus packaging” and “virus production” are used interchangeably. Various methods of producing rAAV are known in the art. The term “packaging system” generally comprises: a plasmid comprising an expression cassette comprising a polynucleotide encoding a molecule of interest, a plasmid comprising polynucleotides encoding AAV structural and non-structural proteins, and other components which assist in rAAV production. In some embodiments, the packaging system comprises a helper virus. rAAVs are replication-defective viruses, that is, they lack the ability to replicate on their own. Helper viruses enable the replication of rAAV by providing components which help the rAAV replicate. Exemplary embodiments of helper viruses include, without limitation, adenovirus (Ad) and herpes simplex virus (HSV). By way of example, plasmids comprising the polynucleotide encoding the gene of interest, AAV rep, and AAV cap genes may be transfected into cells already comprising Ad. The cells are then maintained to allow for rAAV production before harvesting the rAAV. In some embodiments, the packaging system may comprise a two-plasmid AAV packaging system. For example, the expression cassette may be cloned into one plasmid and the AAV rep, cap genes and helper virus genes may be cloned into a second plasmid. The term “helper virus gene” as used herein relates to all the DNA sequences of helper viruses which are necessary for AAV production. The plasmids are transfected into cells suitable for rAAV production. In alternate embodiments, the packaging system may comprise a three-plasmid AAV packaging system. For example, the expression cassette may be cloned into a first plasmid, the AAV rep and cap genes may be cloned into a second plasmid, and the helper virus genes cloned into a third plasmid. The three plasmids are transfected into a cell expression system for the production of rAAVs. In other embodiments, a packaging system comprising a baculovirus is also contemplated. Baculoviruses are pathogens that attack insects and other arthropods. By way of example, the expression cassette, AAV rep, and AAV cap genes may be cloned into baculovirus plasmids. These plasmids are then transfected into insect cells such as Sf9 cells, in which baculovirus are produced. The baculovirus is then used to infect insect cells such as Sf9 cells, in which rAAVs are produced. The baculovirus packaging system possesses many advantages, including the ease of scaling up production and the ability of insect cells to grow in serum-free media.

Aspects of this disclosure include a cell comprising an AAV packaging plasmid system described herein. As will be appreciated by one skilled in the art, the AAV packaging plasmid system may be used to transfect any cell system suitable for the production of rAAVs. In some embodiments, the cells comprise bacteria cells, mammalian cells, yeast cells, or insect cells. The cells may be suspension cells or adherent cells. Examples of suitable cells include, but are not limited to, an Escherichia coli cell, an HEK293 cell, an HEK293T cell, an HEK293A cell, an HEK293S cell, an HEK293FT cell, an HEK293F cell, an HEK293H cell, a HeLa cell, an SF9 cell, an SF21 cell, an SF900 cell, and a BHK cell.

Disclosed embodiments also include a method of producing a rAAV as described herein. In some embodiments, the method may comprise introducing the packaging plasmid system into cells suitable for rAAV production, culturing the cells under suitable conditions, harvesting the rAAVs produced, and optionally purifying the rAAVs. Methods for purifying rAAVs are known in the art. For example, the rAAVs may be purified using chromatography. “Chromatography” as used herein refers to any of methods known in the art for selectively separating out one or more elements from a mixture. Such methods comprise, but are not limited to, affinity chromatography, ion-exchange chromatography, and size exclusion chromatography known in the art.

Aspects of the disclosure also include an isolated, engineered cell comprising the rAAV disclosed herein. In some embodiments, the engineered cell is an animal cell. In some embodiments, the engineered cell is a mammalian cell. In an embodiment, the engineered cell is a human cell.

### Compositions and Treatment of Diseases

Aspects of this disclosure include compositions comprising the rAAVs disclosed herein. The terms “composition” and “formulation” are used interchangeably herein. In some embodiments, the composition is a therapeutic composition. In some embodiments, the composition comprising the rAAVs may further comprise one or more additional therapeutic agents. In certain embodiments, the composition comprising the rAAVs may further comprise one or more pharmaceutically acceptable excipients and/or diluents. Although the descriptions of compositions provided herein are principally directed to compositions which are suitable for administration to humans, it will be understood by the skilled artisan that such compositions are generally suitable for administration to any other animal. Formulations of this disclosure may comprise, without limitation, saline, liposomes, lipid nanoparticles, polymers, peptides, proteins, cells infected with the rAAVs, and combinations thereof.

Compositions disclosed herein may be prepared using any method known in the art. In some embodiments, compositions of the present disclosure are aqueous formulations (i.e. formulations which comprise water). In certain embodiments, formulations of the present disclosure comprise water, sanitized water, or Water-for-injection (WFI). In some embodiments, the rAAVs may be formulated in PBS. In certain embodiments, the rAAV formulations may comprise a buffering system. Exemplary examples of buffering systems include, but are not limited to, buffers comprising phosphate, Tris and/or histidine.

In accordance with some embodiments of this disclosure, the composition disclosed herein may comprise one or more excipients and/or diluents. As will be appreciated by a skilled artisan, the presence of excipients and/or diluents may confer certain advantages, including (1) increased stability; (2) increased cell transfection or transduction; (3) sustained or delayed release of the therapeutic encoded by the transgene; (4) alteration of the biodistribution (e.g., target the virus to specific tissues or cell types); (5) increased translation of the protein encoded by the transgene; (6) altered release profile of protein encoded by the transgene, and/or (7) regulatable expression of the transgene of the present disclosure. Excipients, as used herein, comprise, but are not limited to, any or/and all solvents, dispersion media, or other liquid vehicles, dispersion or suspension aids, surface-active agents, isotonic agents, thickening or emulsifying agents, preservatives, and the like, as suited to the particular dosage form desired. In some embodiments, the composition may comprise a surfactant, including anionic, zwitterionic, or non-ionic surfactants. Surfactants may help control shear forces in suspension cultures.

Aspects of this disclosure also contain compositions comprising various concentrations of rAAVs, which may be optimized according to the characteristics of the formulation and its application. For example, the concentration of rAAV particles may be between about 1×10⁶ VG (vector genomes)/mL and about 1×10¹⁸ VG/mL. In certain embodiments, the formulation may comprise a rAAV particle concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, 1×10¹⁷, 2×10¹⁷, 3×10¹⁷, 4×10¹⁷, 5×10¹⁷, 6×10¹⁷, 7×10¹⁷, 8×10¹⁷, 9×10¹⁷, or 1×10¹⁸VG/mL.

In some embodiments, the concentration of rAAVs in the composition may be between about 1×10⁶ VG/mL and about 1×10¹⁸ total VG/mL. In certain embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, 1×10¹⁷, 2×10¹⁷, 3×10¹⁷, 4×10¹⁷, 5×10¹⁷, 6×10¹⁷, 7×10¹⁷, 8×10¹⁷, 9×10¹⁷, or 1×10¹⁸ total VG/mL.

Other aspects contemplated in this disclosure are the total dose of rAAVs in a composition, e.g., in a vial of formulated product for administration to a patient. In some embodiments, the composition may comprise a total dose of rAAVs of between about 1×10⁶ VG and about 1×10¹⁸ VG. In certain embodiments, the formulation may comprise a total dose of rAAVs of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, 1×10¹⁷, 2×10¹⁷, 3×10¹⁷, 4×10¹⁷, 5×10¹⁷, 6×10¹⁷, 7×10¹⁷, 8×10¹⁷, 9×10¹⁷, or 1×10¹⁸ VG.

Consistent with embodiments of this disclosure, methods of treating a disease in a patient in need thereof, comprising administering a therapeutically effective amount of the rAAV or composition disclosed herein are contemplated. In some embodiments, the methods involve gene therapy. As used herein, the term “gene therapy” generally refers to the utilization of delivery of therapeutic nucleic acids into the cells of a subject to treat a disease. In other embodiments, the use of the rAAV or composition disclosed herein for the preparation of a medicament for treating a disease in a patient is also contemplated. As used herein, the term “patient” may refer to a subject with a disease or other affliction. A patient may be a human or any other animal. In some embodiments, the disease is a liver disease. In certain embodiments, the liver disease is Hepatitis B or Fabry disease.

By way of example, the rAAV of this disclosure may comprise an expression cassette comprising a polynucleotide sequence encoding an shRNA useful for the treatment of Hepatitis B, or GLA, which is useful in the treatment of Fabry disease. In some embodiments, administration of the rAAV comprising GLA results in an increased level of GLA expression in tissue compared to a corresponding rAAV comprising an AAV8 capsid protein. In other embodiments, administration of the rAAV comprising an shRNA useful in treating Hepatitis B results in increased inhibition of Hepatitis B surface antigen (HBsAg), Hepatitis B e-antigen (HBeAg), and/or HBV DNA, compared to a corresponding rAAV comprising an AAV8 capsid protein. As used herein, the term “corresponding rAAV” refers to a rAAV which only differs from the rAAV of interest in the capsid protein.

The rAAV or compositions disclosed herein may be administered via any method known in the art. In some embodiments, the rAAV or composition may be administered via intravenous administration. In certain embodiments, the rAAV or composition may be administered through infusion or injection. In certain embodiments, the rAAV or composition may be administered via parenteral injection. Other administration methods include, but are not limited to, subcutaneous, intravenous, intraperitoneal, or intramuscular injection. In a particular embodiment, the rAAV or composition is administered via intravenous injection. In some embodiments, the rAAV or composition may be administered in a single dose. In alternate embodiments, the rAAV or composition may be administered in multiple dosages.

Consistent with embodiments of this disclosure, methods of treating a disease in a patient may comprise administration of a second active agent in addition to the rAAV or compositions disclosed herein. In some embodiments, the second active agent may be administered simultaneously. In alternate embodiments, the second active agent may be administered sequentially. By way of example, a method of treating a patient with Hepatitis B may comprise administration of Lamivudine and/or Entecavir in addition to the administration of the pharmaceutical composition disclosed herein.

### EXAMPLES

### Example 1: Plasmid Construction

Construction of pSC-DC172-Gluc, pSC-DC190-Gluc, and pSC-CMV-Gluc Plasmids

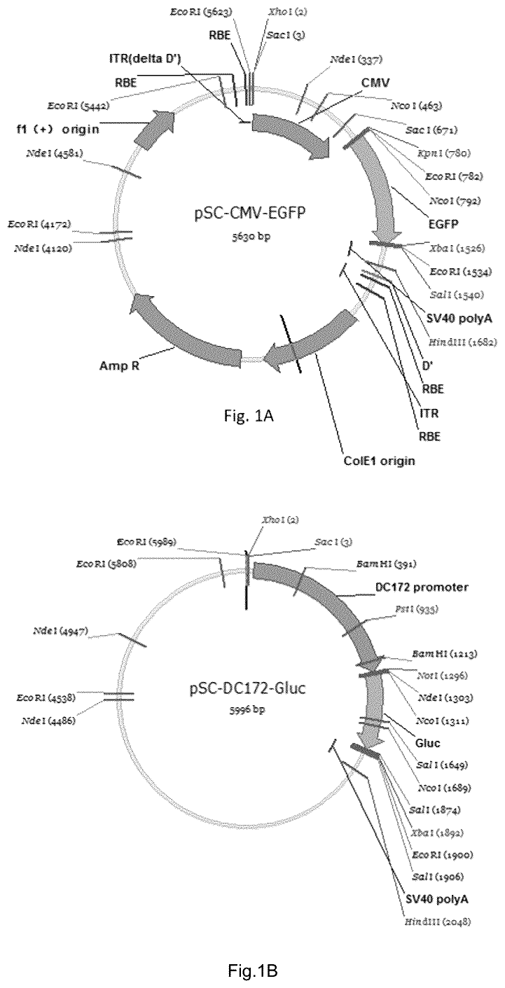

Three sequence fragments containing the sequence of DC172 promoter (SEQ ID NO: 20), the sequence of DC190 promoter (SEQ ID NO: 22), the sequence of CMV promoter respectively, each of which is flanked by XhoI and NotI restriction sites, were synthesized and digested with XhoI and NotI restriction enzymes separately. The sequence of Gaussia luciferase (Gluc) which is flanked by NotI and XbaI restriction sites was synthesized and digested with NotI and XbaI restriction enzymes. The plasmid pSC-CMV-EGFP (FIG. 1A) was digested with XhoI and XbaI restriction enzymes and ligated with the digested DC172 and Gluc fragments to generate plasmid pSC-DC172-Gluc (FIG. 1B). The plasmid pSC-CMV-EGFP (FIG. 1A) was digested with XhoI and XbaI restriction enzymes and ligated with the digested DC190 and Gluc fragments to generate plasmid pSC-DC190-Gluc (FIG. 1C). The pSC-CMV-EGFP (FIG. 1A) was digested with XhoI and XbaI restriction enzymes and ligated with the digested CMV and Gluc fragment to generate plasmid pSC-CMV-Gluc (FIG. 1D). These plasmids were used to produce self-complementary AAVs (scAAVs).

Construction of pSNAV2.0-DC172-GLA, pSNAV2.0-DC172-GLA-wpre, pSNAV2.0-LP1-GLA, and pSNAV2.0-LP1-GLA-wpre Plasmids

Three sequence fragments containing LP1 promoter (SEQ ID NO: 21) with flanking XhoI and NotI restriction sites, alpha-galactosidase (GLA, GeneBank NM_000169.2) with flanking NotI and SalI restriction sites, and the Woodchuck Hepatitis Virus Posttranscriptional Regulatory Element (WPRE/wpre, SEQ ID NO: 8) with flanking SalI restriction sites, respectively, were synthesized, followed by digestion with the respective restriction enzymes. pSNAV2.0-EGFP (FIG. 1E) was digested with XhoI and SalI and ligated with the DC172 digested with XhoI and NotI and GLA fragments digested with NotI and SalI to generate plasmid pSNAV2.0-DC172-GLA (FIG. 1F). pSNAV2.0-DC172-GLA was digested with SalI and ligated with the digested WPRE fragment to generate plasmid pSNAV2.0-DC172-GLA-wpre (FIG. 1G). pSNAV2.0-EGFP was digested with XhoI and SalI and ligated with the LP1 digested with XhoI and NotI and GLA fragments digested with NotI and SalI to generate plasmid pSNAV2.0-LP1-GLA (FIG. 1H). pSNAV2.0-LP1-GLA was digested with SalI and ligated with the digested WPRE fragment to generate plasmid pSNAV2.0-LP1-GLA-wpre (FIG. 1I). These plasmids were used to produce single-stranded AAVs (ssAAVs).

Construction of pSC-H1-shRNA-intron2 Plasmid

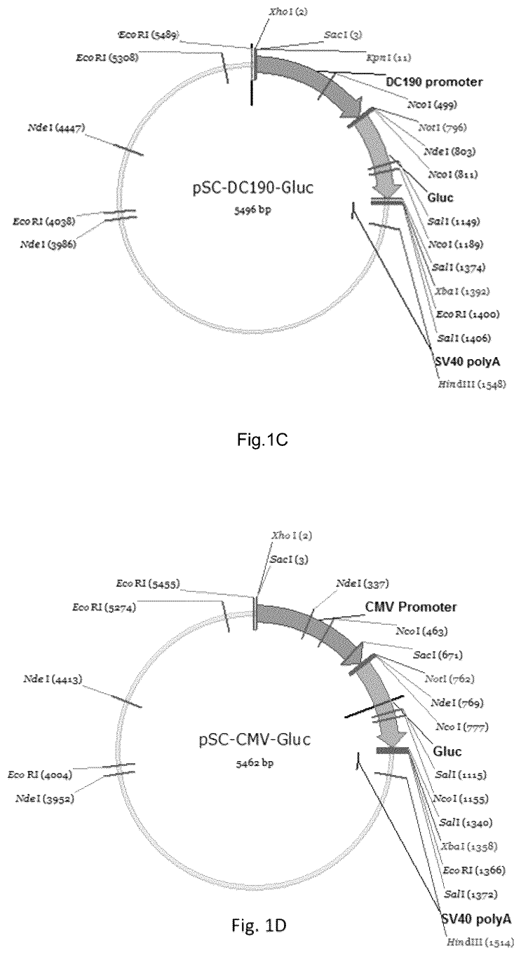

pSC-CMV-EGFP, a shuttle vector for producing self-complementary AAVs, was constructed and retained the ITR derived from AAV2. This vector was digested with BglII and XhoI restriction enzymes and ligated with DNA sequences comprising H1 promoter (SEQ ID NO: 9) and polynucleotide sequence that encodes shRNA targeting HBV (SEQ ID NO: 3) digested with BglII and XhoI restriction enzymes to replace the CMV-EGFP fragment in the vector to form a new plasmid pSC-H1-shRNA (FIG. 2A). Intron2 from HPRT-intron (positions 1846-3487 of GenBank: M26434.1) was synthesized, digested with BglII and HindIII restriction enzymes, and inserted into the pSC-H1-shRNA vector digested with BglII and HindIII restriction enzymes to generate plasmid pSC-H1-shRNA-intron2 (FIG. 2B).

### Example 2: Virus Production and Assessment of Viral Titer

Recombinant AAVs used in the experiments were produced in HEK293 cells (obtained from ATCC) by the known three-plasmid packaging system via standard techniques (Xiao et al., Production of high-titer recombinant adeno-associated virus vectors in the absence of helper adenovirus. J. Virol. 72(3): 2224 (1998)). Suitable amounts of purified virus samples were added into a digestion reaction solution described in Table 1, incubated at 37° C. for 30 min, and further incubated at 75° C. for 10 min to deactivate the DNase I. The digested AAV was diluted and analyzed using qPCR, as shown in Table 2.

TABLE 1
DNase I digestion of AAV sample

| | | |
|---|---|---|
| AAV sample | 5 | μL |
| 10 × DNase I buffer | 5 | μL |
| DNase I | 1 | μL |
| RNase-free water | 39 | μL |
| Total | 50 | μL |

TABLE 2
qPCR protocol

| Reaction Mixture | | | qPCR protocol | | |
|---|---|---|---|---|---|
| Sample | 5 | μL | 50° C. 2 min | 1 | cycle |
| Forward Primer (10 μM) | 0.5 | μL | 95° C. 10 min | | |
| Reverse Primer (10 μM) | 0.5 | μL | 95° C. 15 sec | 40 | cycles |
| Probe Primer (10 μM) | 0.5 | μL | 60° C. 30 sec | | |
| Taqman PCR Mix (2×) | 10 | μL | 37° C. 1 sec | 1 | cycle |

| | | |
|---|---|---|
| ddH₂O | 3.5 | μL |

Primers used for the qPCR are shown in Table 3. Viral titers as measured by qPCR are shown in Table 4.

TABLE 3
Primers used for the qPCR

| | |
|---|---|
| Gluc Forward primer | CGAGAACAACGAAGACTTCAACA |
| | (SEQ ID NO: 11) |
| Gluc Reverse primer | CGCGGTCAGCATCGAGAT |
| | (SEQ ID NO: 12) |
| Gluc Probe primer | CCGTGGCCAGCAACTTCGCG |
| | (SEQ ID NO: 13) |
| GLA Forward primer | CTGCCAGGAAGAGCCAGATT |
| | (SEQ ID NO: 14) |
| GLA Reverse primer | GTACTCATAACCTGCATCCTTCCA |
| | (SEQ ID NO: 15) |
| GLA Probe primer | TGCATCAGTGAGAAGC |
| | (SEQ ID NO: 16) |
| H1 Forward primer | ATCAACCCGCTCCAAGGAAT |
| | (SEQ ID NO: 17) |
| H1 Reverse primer | AACACATAGCGACATGCAAATATTG |
| | (SEQ ID NO: 18) |
| H1 Probe primer | CCCAGTGTCACTAGGCGGGAACACC |
| | (SEQ ID NO: 19) |

TABLE 4
Viral titers measured by qPCR

| Plasmid | Viral Vector | Titer (vg/mL) |
|---|---|---|
| pSC-DC172-Gluc | scAAV2/X- DC172-Gluc | 1.98E+10 |
| pSC-DC190-Gluc | scAAV2/X- DC190-Gluc | 5.71E+10 |
| pSC-CMV-Gluc | scAAV2/X- CMV-Gluc | 2.72E+10 |
| pSNAV2.0-DC172-GLA | ssAAV2/X- DC172-GLA | 1.82E+11 |
| pSNAV2.0-DC172-GLA-wpre | ssAAV2/X- DC172-GLA-WPRE | 2.1E+11 |
| pSNAV2.0-LP1-GLA | ssAAV2/X- LP1-GLA | 1.57E+11 |
| | ssAAV2/8- LP1-GLA | 2.61E+11 |
| pSNAV2.0-LP1-GLA-wpre | ssAAV2/X- LP1-GLA-WPRE | 1.6E+11 |
| pSC-H1-shRNA-intron2 | scAAV2/8-H1-shRNA-intron2 | 5.50E+12 |
| | scAAV2/X-H1-shRNA-intron2 | 1.12E+12 |
| pSC-CMV-EGFP | scAAV2/8-CMV-EGFP | 3.15E+11 |
| | scAAV2/X-CMV-EGFP | 2.87E+11 |
| | scAAV2/3B-CMV-EGFP | 1.15E+12 |
| | scAAV2/7-CMV-EGFP | 5.64E+12 |
| | scAAV2/9-CMV-EGFP | 3.87E+12 |
| | scAAV2/2-CMV-EGFP | 6.87E+11 |

### Example 3: AAV Vector Selection

### 3.1 Comparison of AAV2/8, AAV2/3B, AAV2/7, and AAV2/9 Tropism for Various Liver Cell Lines In Vitro

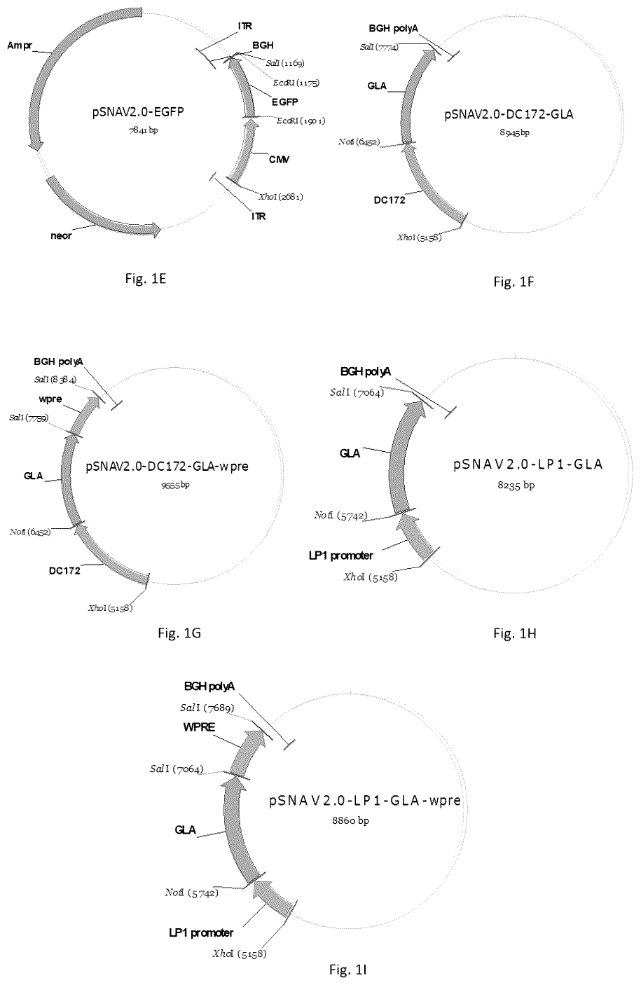

Six different liver cells lines (HepG2, Huh-7, 7402, 7721, Huh-6, and L-02) were infected with scAAV2/8-CMV-EGFP, scAAV2/3B-CMV-EGFP, scAAV2/7-CMV-EGFP, or scAAV2/9-CMV-EGFP, respectively, and infection efficiency was analyzed and compared using flow cytometry. As shown in FIG. 3A-FIG. 3F, at different MOI, AAV2/3B exhibited the same infection efficiencies on HepG2 as AAV2/8. At different MOI, AAV2/3B exhibited higher infection efficiencies on Huh-7 cell lines than AAV2/8. For the remaining four cell lines, at different MOI, the infection efficiencies of AAV2/8 were greater than those of AAV2/3B.

### 3.2 Comparison of AAV2/8 and AAV2/X Tropism for Various Liver Cell Lines In Vitro

AAV2/8 exhibits the greatest tropism for liver cells according to the experiments of Example 3.1. AAVX is a recombinant AAV with the capsid produced by using DNA shuffling technology. The tropism of AAV2/8 and AAV2/X for liver cells was compared. Five liver cell lines (Huh-6, 7402, Huh-7, HepG2, and 7721) was infected with scAAV2/8-CMV-EGFP or scAAV2/X-CMV-EGFP, respectively, and infection efficiency was analyzed and compared using flow cytometry. The comparison involves comparing the differences of MOI needed for the two different AAVs to achieve the same infection efficiency in the same cell line.

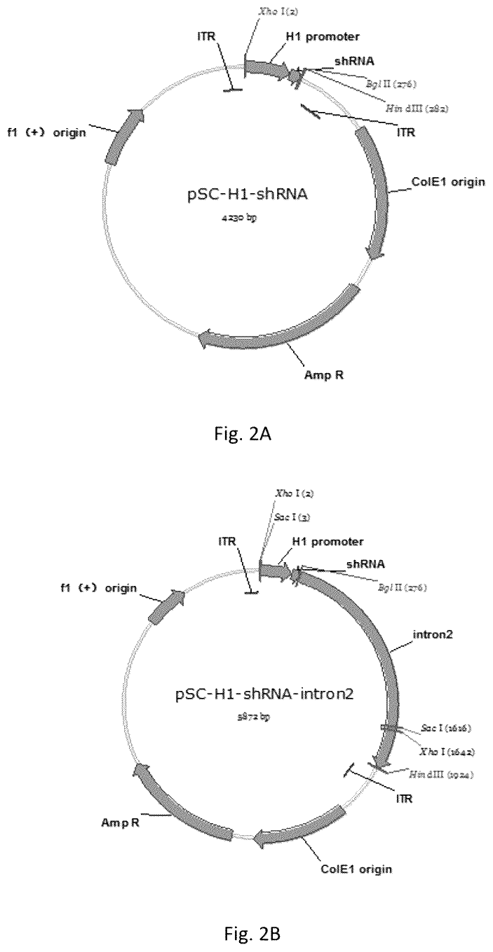

In multiple liver cell lines, the infection efficiency of scAAV2/X-CMV-EGFP was substantially higher than that of scAAV2/8-CMV-EGFP. To achieve the same infection efficiency in Huh-6 cells, the MOI of AAV2/8-CMV-EGFP was about three times that of AAV2/X-CMV-EGFP. To achieve the same infection efficiency in 7402 cells, the MOI of AAV2/8-CMV-EGFP was about 10 times that of AAV2/X-CMV-EGFP. To achieve the same infection efficiency in Huh-7 cells, the MOI of AAV2/8-CMV-EGFP was about 100-300 times that of AAV2/X-CMV-EGFP. To achieve the same infection efficiency in HepG2 cells, the MOI of AAV2/8-CMV-EGFP was about 30-100 times that of AAV2/X-CMV-EGFP. To achieve the same infection efficiency in 7721 cells, the MOI of AAV2/8-CMV-EGFP was about 30-100 times that of AAV2/X-CMV-EGFP. The results are shown in FIGS. 4A-4J. These results consistently show substantially higher infection efficiency of AAV2/X compared to AAV2/8 and demonstrate superior tropism of AAV2/X over AAV2/8 in a variety of liver cell lines.

### 3.3 Comparison of AAV2/8 and AAV2/X Tropism for Human Primary Liver Cell

The tropism of AAV2/8 and AAV2/X for primary liver cells derived from human HBV patients was further evaluated through infection assays. Primary liver cells from five liver cancer patients (HCC307N1, HCC061A2, HCC213F1, HCC893D1, HCC554A4; shown in Table 5) were isolated. The cells were then cultured and infected with scAAV2/8-CMV-EGFP or scAAV2/X-CMV-EGFP. MOIs of 5000, 15000, 50000, 150000, or 500000 of scAAV2/8-CMV-EGFP or scAAV2/X-CMV-EGFP was used for HCC307N1 cells; MOIs of 5000, 15000, 50000, 150000, or 500000 of AAV2/8-CMV-EGFP was used for HCC061A2 cells and MOIs of 500, 1500, 5000, 15000, or 50000 of AAV2/X-CMV-EGFP was used for HCC061A2 cells; MOIs of 500, 1500, 5000, 15000, 50000, 150000, or 500000 for AAV2/8-CMV-EGFP or AAV2/X-CMV-EGFP was used for the three remaining cells lines. Forty-eight hours after infection, the images of infected cells were detected by fluorescence microscope, and the percentages of GFP-positive cells and fluorescence intensities were measured by flow cytometry.

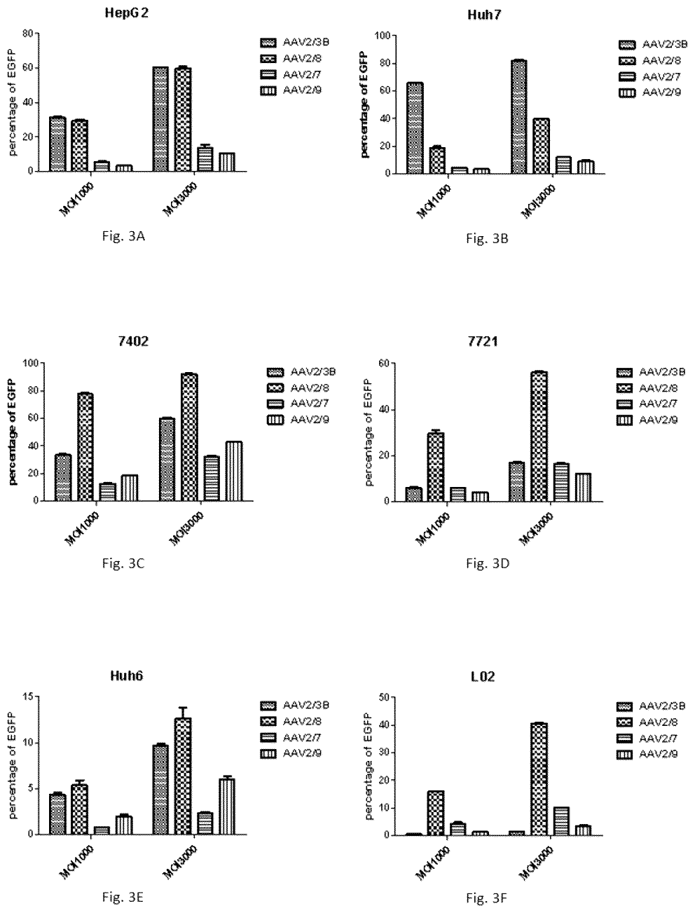

FIGS. 5A-5J show that under the same MOI condition, infection with scAAV2/X-CMV-EGFP resulted in a higher percentage of GFP-positive cells and a greater intensity of GFP fluorescence compared to infection with scAAV2/8-CMV-EGFP. These results in primary liver cells, in consistent with the liver cell lines experiments, show that AAV2/X has superior tropism for liver cells compared to AAV2/8 in primary liver cell.

TABLE 5
Clinical parameters of study patients

| | Patient Clinical Parameters | | | | | | | HBV |
|---|---|---|---|---|---|---|---|---|
| | | | | | | | HBV | Integration |
| | | | | | HBsAg | HBeAg | DNA | into |
| Patient ID | Sex | Age | Indication | Stage | Presence | Presence | concentration | Genome |
| HCC307N1 | male | 34 | HCC* | I | + | − | 2850 | + |
| HCC061A2 | male | 56 | HCC | I | + | + | 168000 | + |
| HCC213F1 | male | 40 | HCC | IVa | + | + | 2510000 | + |
| HCC893D1 | male | 47 | HCC | II | + | + | Lower than | + |
| | | | | | | | detection | |
| | | | | | | | limit | |
| HCC554A4 | male | 51 | HCC | II | + | - | 1340 | + |

*Denotes hepatocellular carcinoma

### 3.4 In Vivo Tropism Comparison of AAV2/8 and AAV2/X in Macaques

Six macaques (three males and three females) were divided into two groups, with each group containing three animals. Whereas scAAV2/8-CMV-EGFP was administered to one group of animals through intravenous injection at a single dose of 1E+12 vg/kg, scAAV2/X-CMV-EGFP was administered to the other group of animals through intravenous injection at a single dose of 1E+12 vg/kg. The animals were euthanized seven days after administration and tissues from heart, lung, liver, brain, testicle, ovary, thigh bicep, stomach, jejunum, kidney, and spleen were collected and analyzed for GFP expression using fluorescence microscopy.

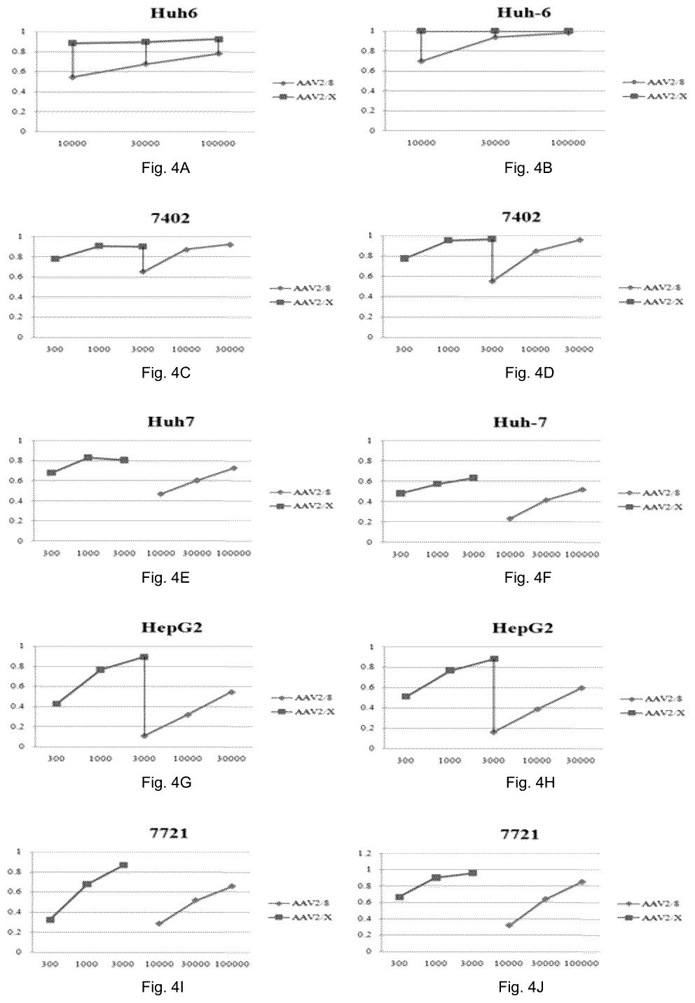

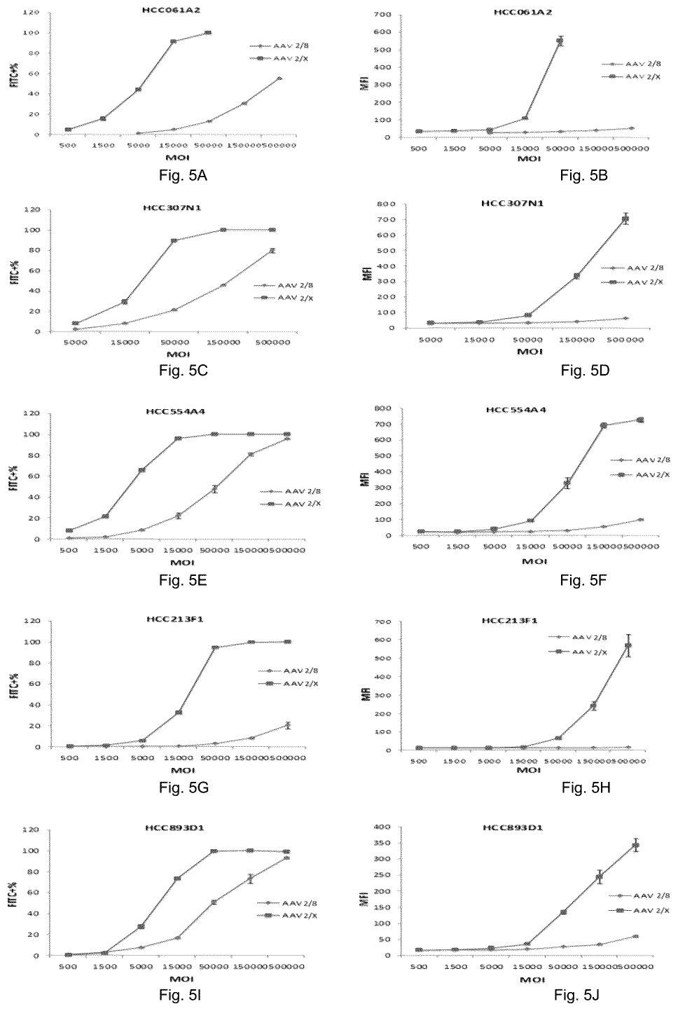

GFP expression was readily visible in the liver tissues of the tested animals. Results are shown in FIGS. 6A-6N and 7A-7N and Table 6. The number of GFP-positive cells per microscopy field of view from the liver tissues of three animals administered with scAAV2/8-CMV-EGFP were 15.00±4.47, 8.20±2.39, and 8.00±5.83, respectively. The number of GFP-positive cells per microscopy field of view from the liver tissues of three animals administered with scAAV2/X-CMV-EGFP were 123.40±8.02, 79.80±23.06, and 54.40±28.01, respectively. GFP expression levels in different liver tissues from the same animal were not substantially different. These results show that the numbers of GFP-positive cells from macaques that were administered with scAAV2/X-CMV-EGFP were statistically significantly higher than that from macaques that were administered with scAAV2/8-CMV-EGFP. The in vitro and in vivo results from the experiments discussed above fully demonstrate that AAV2/X has superior tropism for the liver over AAV2/8 both in vitro and in vivo.

TABLE 6
Fluorescence microscopy of GFP expression

| | | Liver | | | | | | |
|---|---|---|---|---|---|---|---|---|
| Group | Animal ID | 1 | 2 | 3 | 4 | 5 | Mean | SD |
| SCAAV2/ | 1~1 | 21 | 13 | 9 | 17 | 15 | 15.00 | 4.47 |
| 8-CMV-EGFP | 1~2 | 12 | 9 | 6 | 7 | 7 | 8.20 | 2.39 |
| | 1~3 | 18 | 8 | 6 | 4 | 4 | 8.00 | 5.83 |
| SCAAV2/ | 2~1 | 113 | 131 | 126 | 130 | 117 | 123.40 | 8.02 |
| X-CMV-EGFP | 2~2 | 63 | 83 | 100 | 50 | 103 | 79.80 | 23.06 |
| | 2~3 | 98 | 61 | 29 | 53 | 31 | 54.40 | 28.01 |

### Example 4: Comparison of Levels of Neutralizing Antibodies (Nab) Against Different AAV Serotypes in Serum

### 4.1 Comparison of Levels of Nab Against AAV2/8, AAV2/3B, and AAV2/2 in Human Serum

This experiment adopts a method that uses a fixed viral MOI and serially diluted serum. The virus MOI was 10000. The serially diluted serum and virus were mixed at a 1:1 ratio and incubated at 37° C. for one hour. HepG2 cells were cultured in 24-well plates for 24 hours and then infected with adenovirus Ad5. After two hours, the virus-containing media was removed and the cells were washed with DPBS. The diluted serum and virus mixture or virus only (with the same MOI) was then added to the cells and incubated for 48 hours. Following incubation, the cells were collected and analyzed using flow cytometry. The levels of Nabs were calculated by taking the reciprocal of a serum dilution selected from serial dilutions that achieved a cell infection efficiency that is 50% of the cell infection efficacy achieved by the rAAV without serum (Lochrie M A et al. (2006) Virology 353: 68-82; Mori S et al. (2006) Jpn J Infect Dis 59: 285-293).

Results are shown in Table 7. Table 7 shows the Nab results of serum from human individuals. Among the serum from 13 individuals, AAV2/8 Nab levels were the lowest. AAV2/3B Nab levels were around 10 times greater than AAV2/8 Nab levels. In 11 samples, levels of AAV2/2 Nab were equal to, or lower than, levels of AAV2/3B Nab. In two samples, the levels of AAV2/2 Nab were greater than AAV2/3B Nab levels. The results show that the levels of Nab of AAV2/8 were substantially lower (over 10-fold lower than that of AAV2/3B). These results demonstrate that AAV2/8 is more superior over AAV2/3B as a gene therapy vector and AAV2/8 is less immunogenic in humans compared to other AAV serotypes such as AAV2/3B or AAV2/2.

TABLE 7
Nab in serum from individuals

| Samples | AAV2/2 | AAV2/3B | AAV2/8 |
|---|---|---|---|
| No. 1 | 40-80 | 80-160 | <8 |
| No. 2 | >160 | >160 | 8-16 |
| No. 3 | 40-80 | 80 | 8-16 |
| No. 4 | 10-20 | 20-40 | <2 |
| No. 5 | >160 | 80-160 | 8-16 |
| No. 6 | <10 | 10-20 | <2 |
| No. 7 | 20-40 | 80-160 | 4-8 |
| No. 8 | >160 | >160 | 32 |
| No. 9 | 10-20 | 40-80 | 4-8 |
| No. 10 | 40-80 | 40-80 | 4-8 |
| No. 11 | 80-160 | 80-160 | 8-16 |
| No. 12 | 40-80 | 40-80 | 2-4 |
| No. 13 | >160 | 40-80 | 2-4 |

### 4.2 Comparison of Levels of Nab Against AAV2/8 and AAV2/X in Macaque Serum

Serum from 12 macaques were utilized to determine the levels of Nab against AAV2/X and AAV2/8, using the protocol described above. The virus MOI was 2000. The serially diluted macaque serum and scAAV2/X-CMV-EGFP or scAAV2/8-CMV-EGFP were mixed at a 1:1 ratio and incubated at 37° C. for one hour. The 7402 cell line was cultured in 24-well plates. The diluted macaque serum and virus mixture or virus only (with the same MOI) was then added to the cells and incubated for 48 hours. Following incubation, the cells were collected and analyzed using flow cytometry.

The serum samples were diluted in 4 series, with a dilution range of 5 to 100 fold. Nab amount lower than 5 is deemed negative. The twelve samples are numbered individually from 1 #to 12 #. Results are shown in Table 8. Samples 1 #, 2 #, and 4 #showed no Nabs against either AAV serotype. Sample 7 #exhibited relatively low levels of Nab against both serotypes. Samples 3 #and 12 #showed greater levels of Nab against scAAV2/8 and tested negative for Nab against scAAV2/X. Samples 5 #, 6 #, 8 #, 9 #, 10 #, and 11 #exhibited substantially lower levels of scAAV2/X Nab compared to scAAV2/8 Nab. These results show that levels of AAV2/X Nab were lower than AAV2/8 Nab in macaques. These results demonstrate that AAV2/X exhibits lower immunogenicity as superior gene delivery vectors.

TABLE 8
Nab levels of AAV2/X and AAV2/8 in macaque serum

| Sample | | |
|---|---|---|
| No. | AAV2/X Nab | AAV2/8 Nab |
| 1# | <5 | <5 |
| 2# | <5 | <5 |
| 3# | ≤5 | >100 |
| 4# | <5 | <5 |
| 5# | 10-50 | >100 |
| 6# | 10-50 | >100 |
| 7# | 10 | 10 |
| 8# | 10-50 | >100 |
| 9# | 50-100 | >100 |
| 10# | 50-100 | >100 |
| 11# | 50-100 | >100 |
| 12# | <5 | 10-50 |

### 4.3 Comparison of Levels of Nab Against AAV2/8 and AAV2/X in Human Serum

In these experiments, levels of Nabs against AAV2/8 and AAV2/X in serum from healthy human individuals were examined and compared after cells were infected with rAAV with fixed MOI. The levels of Nabs were calculated by taking the reciprocal of a serum dilution factor selected from serial dilutions that achieved a cell infection efficiency that is 50% of the cell infection efficacy achieved by the rAAV without serum. Serum from 20 human individuals was diluted serially and analyzed for Nab against AAV2/X and AAV2/8, using the protocol described above. The 7402 cell line was cultured in 24-well plates. Human serum samples were serially diluted. scAAV2/8-CMV-EGFP or scAAV2/X-CMV-EGFP with MOI of 2000 were added to the serially diluted serum at a 1:1 ratio and incubated at 37° C. for an hour. The mixture or scAAV2/8-CMV-EGFP or scAAV2/X-CMV-EGFP with MOI of 2000 was then added to the cultured cells and incubated for 48 hours, after which the cells were collected and analyzed using flow cytometry.

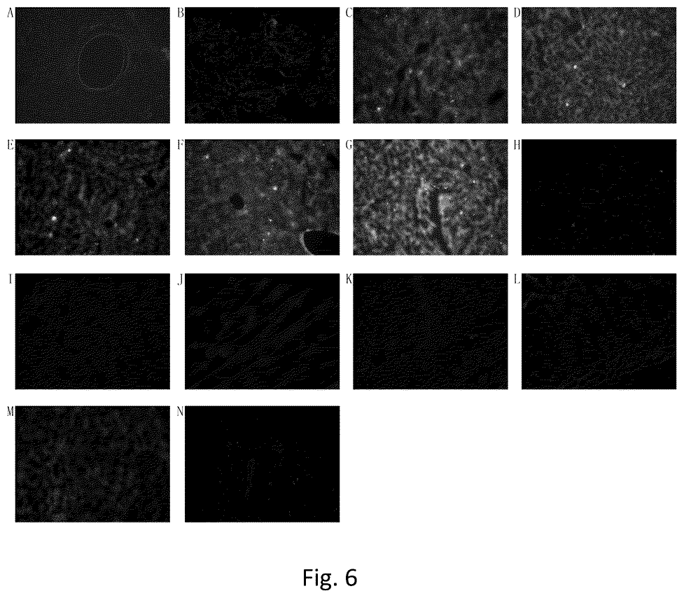

Results are shown in FIG. 8 and Table 9. The results show that in nine serum samples (2 #, 5 #, 6 #, 7 #, 9 #, 15 #, 16 #, 17 #, 20 #), higher levels of Nab against AAV2/8 were observed compared to Nab against AAV2/X. In four serum samples (4 #, 13 #, 14 #, 18 #), higher levels of Nab against AAV2/X were observed compared to Nab against AAV2/8. In three samples (1 #, 3 #, 8 #), the levels of Nab against AAV2/8 were similar to levels of Nab against AAV2/X. Four serum samples (10 #, 11 #, 12 #, 19 #) were tested negative for both Nab against AAV2/X and Nab against AAV2/8. These in vitro infection experiments compared the amount of Nabs for AAV2/8 and AAV2/X in humans from 20 randomly selected samples, and the results are representative of those in a larger population.

These results confirm that AAV2/X has enhanced tropism for liver cells and lower immunogenicity in humans compared to AAV2/8, making it more suitable for use to deliver a therapeutic.

TABLE 9
Nab levels of AAV2/X and AAV2/8 in human serum

| Sample | AAV2/X-Nab | AAV2/8-Nab |
|---|---|---|
| 1# | 300-400 | 300-400 |
| 2# | 200-300 | 400 |
| 3# | 50 | 40-50 |
| 4# | >30 | 20-30 |
| 5# | 100-200 | 400 |
| 6# | 200 | 400 |
| 7# | 40-80 | 200 |
| 8# | 20-40 | 20-40 |
| 9# | 50-100 | 100-150 |
| 10# | <5 | <5 |
| 11# | <5 | <5 |
| 12# | <5 | <5 |
| 13# | 20-30 | 10-20 |
| 14# | 5-10 | <5 |
| 15# | 200-300 | >400 |
| 16# | 20-40 | >40 |
| 17# | <50 | 100-200 |
| 18# | 300-400 | 50 |
| 19# | <5 | <5 |
| 20# | 200-300 | >400 |

### Example 5: Pharmacology Experiments

### Example 5.1: AAV2/X for Treatment of Fabry Disease

### Promoter Selection

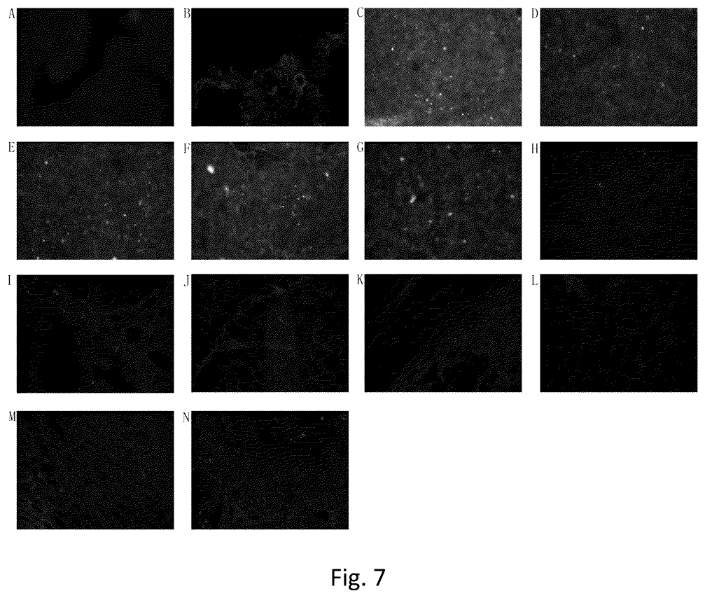

Normal 129 mice were divided into four groups with three animals in each group, including three administration groups and one negative control group. Animals in each administration group were administered via intravenous injection with a dose of 3E+10 vg/animal of scAAV2/X-CMV-Gluc, scAAV2/X-DC172-Gluc, or scAAV2/X-DC190-Gluc, respectively (FIG. 9A). PBS was administered to the negative control group. Blood samples were collected via tail clip one, two, three, and four weeks after administration and analyzed for Gluc expression based on manufacturer's instructions (for example, GaiNing BioPharmaceuticals's Gaussia Luciferase assay kit).

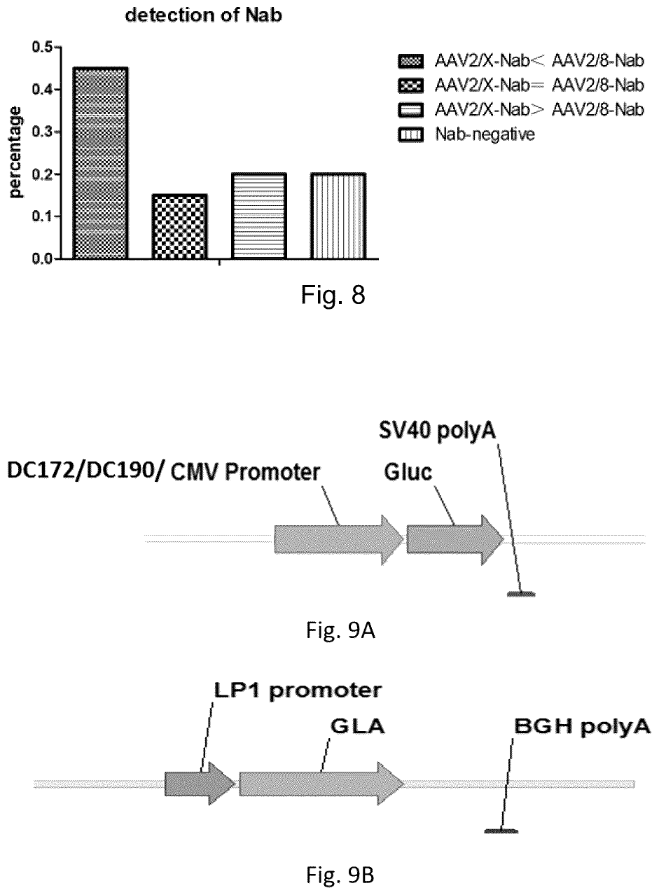

Results show no expression of Gluc in the PBS control animals. Each experimental data point shows that rAAV containing DC172 promoter exhibits the highest level of Gluc expression after infection of normal mice, followed by the rAAV containing DC190 promoter or CMV promoter (FIG. 10A).

The DC172 promoter was then used to construct pSNAV2.0-DC172-GLA, and the LP1 promoter was used to construct pSNAV2.0-LP1-GLA (FIG. 9B), and then recombinant ssAAV2/X-DC172-GLA or ssAAV2/X-LP1-GLA was produced. Recombinant ssAAV2/X-DC172-GLA, ssAAV2/X-LP1-GLA or PBS were administered to three groups of normal mice, respectively, using the protocol described above. The tested dose was 1E+15 vg/animal. Blood was collected via tail clip two, three, four, five, six, seven, and eight weeks after injection and analyzed for GLA activity by a substrate fluorescence method. Specifically, 10 μL serum of each blood sample was added to 96-well fluorescent plates, and 40 μL substrate (5 mM 4-methylumbelliferone-α-D-galactoside (ACROS, 337162500) and 100 mM N-acetyl-D-galactosamine (Sigma, A2795)) were added and mixed well. The mixture was incubated in dark at 37° C. for an hour, and 0.3 M glycine-NaOH was added to stop the reaction. 4-MU (Sigma, M1381) with different molar concentration was used as a standard to calculate the expression level through the degree of fluorescence. Results show that the expression level of GLA driven by the rAAV containing LP1 promoter was higher than that driven by the rAAV containing DC172 promoter in normal mice at each time point. Results are shown in FIG. 10B. Based on the results, the LP1 promoter was selected as the promoter used in the next stage of testing.

### Analysis of Gene Expression of the Woodchuck Hepatitis Virus Posttranscriptional Regulatory Element

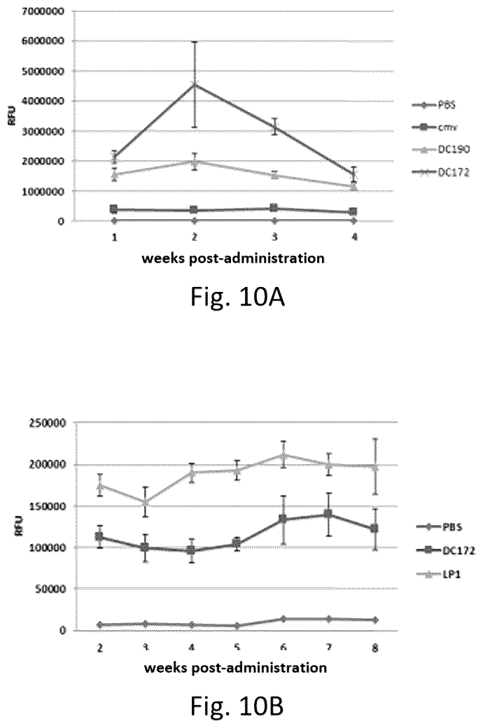

The effects of expression regulatory elements such as WPRE were investigated. ssAAV2/X-DC172-GLA, ssAAV2/X-DC172-GLA-wpre, ssAAV2/X-LP1-GLA, or ssAAV2/X-LP1-GLA-wpre (FIG. 11) were injected into four groups of normal mice, respectively. Each experimental group has three animals, which were administered with the tested rAAV vectors at a dose of 3E+10 vg/animal through tail vein injection. PBS was administered to another group of three mice as a negative control. Blood of the mice was collected via tail clip one, two, three, four, five, six, seven, and eight weeks after administration, and GLA activity was measured.

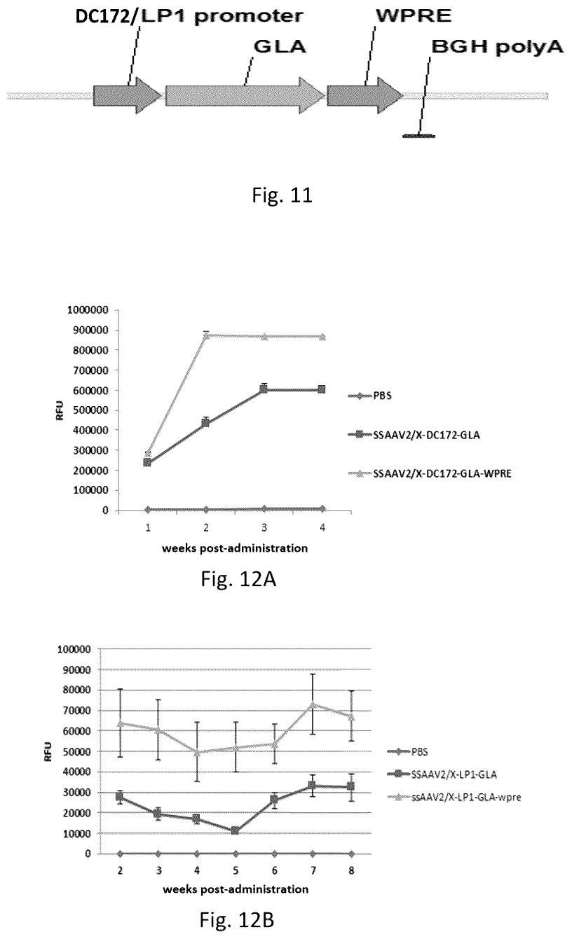

Results show that adding WPRE to ssAAV2/X-DC172-GLA and ssAAV2/X-LP1-GLA increases exogenous gene expression in both instances. The levels of expression of GLA by ssAAV2/X-DC172-GLA-wpre or ssAAV2/X-LP1-GLA-wpre are only 1.5- to 2-fold of that achieved by the viral vectors without WPRE. These results were shown in FIG. 12A-FIG. 12B.

Assessment of Enzyme Activity in Various Tissues of Mice Infected with AAV2/X-LP1-GLA and AAV2/8-LP1-GLA

The relationships between different rAAV administered and GLA expression levels are tested. GLA deficient model Mice (Ohshima T et al. (1997) Proc Natl Acad Sci USA 94(6):2540-2544) were divided into two administration groups that were administered with 1E+10 vg/animal of ssAAV2/X-LP1-GLA or ssAAV2/8-LP1-GLA. Each group contains six animals, including three male mice and three female mice. Two groups of control animals were used in the experiments, including wild-type mice and empty model mice (Gla−/−). Animals were sacrificed seven days after tail vein injection, and serum, liver tissue, heart tissue, and kidney tissue were collected using known techniques, homogenized and centrifuged for 30 min at 4° C. The supernatant was collected and centrifuged again for 10 min at 4° C. Following the final round of centrifugation, the supernatant was collected, and protein concentration was measured with the BCA assay. GLA activity was also measured by a substrate fluorescence method.

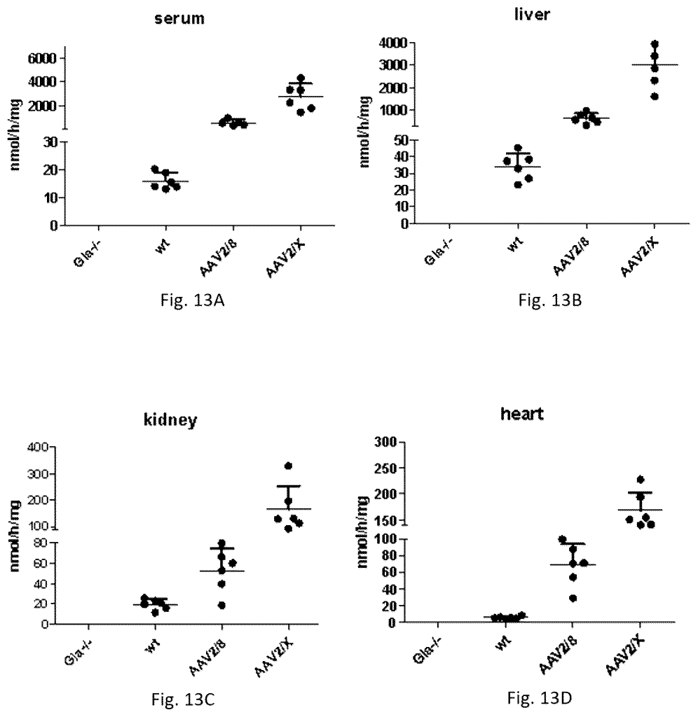

Results show the enzyme activity of GLA in serum, liver, kidney, and heart tissues of model animals infected with the ssAAV2/X-LP1-GLA or ssAAV2/8-LP1-GLA was higher than the enzyme activity of GLA in those tissues of wild-type control mice. Moreover, the enzyme activity of GLA in all tested tissues of model animal infected with ssAAV2/X-LP1-GLA was higher than that of GLA in all tested tissues of model animal infected with ssAAV2/8-LP1-GLA. After systemic administration of the rAAV, the enzyme activity of GLA in serum and the liver tissue is significantly higher than that of GLA in heart and kidney. Thus, although the relatively low doses of rAAV reached kidney and heart through blood circulation, the enzyme activity of GLA was still higher than that in wild-type. These results demonstrate that ssAAV2/X-LP1-GLA achieved superior pharmacological effects compared to ssAAV2/8-LP1-GLA. Results are shown in FIG. 13A-FIG. 13D.

### Analysis of Effect of AAV2/X Viral Titer on GL Activity in Different Tissues

The relationships between doses of rAAV administered and the enzyme activity of GLA are tested. GLA deficient model Mice were divided into three groups that were administered with different doses of ssAAV2/X-LP1-GLA, including 5E+11 vg/kg, 1.5E+11 vg/kg, and 5E+10 vg/kg (about 20 g/mouse). Each group contains six animals, including three male mice and three female mice. Two groups of control animals were used in the experiments, including wild-type mice and empty model mice (Gla−/−). Animals were sacrificed seven days after tail vein injection, and serum, liver tissue, heart tissue, and kidney tissue were collected using known techniques. Tissues were homogenized as described above and protein concentrations were measured using the BCA assay. GLA activity in serum and tissues was measured by a substrate fluorescence method.

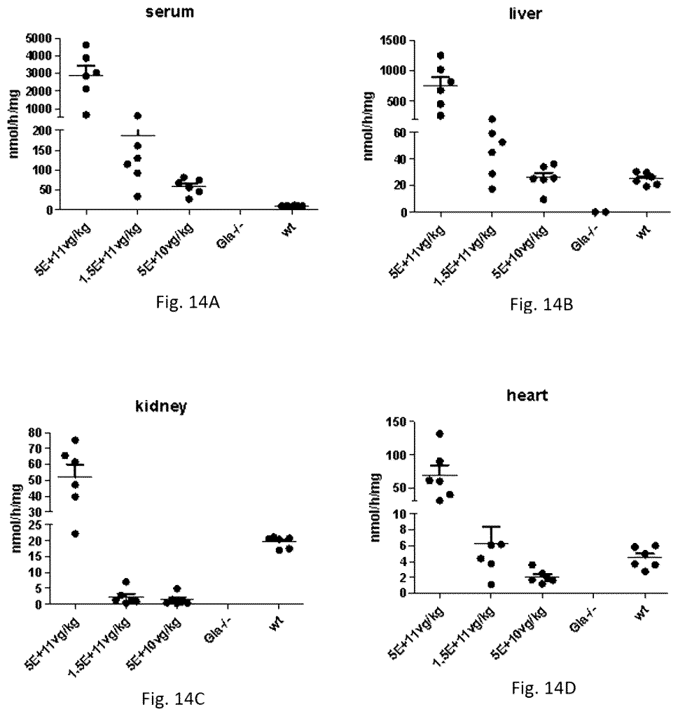

Results show that in serum, higher viral titers resulted in progressively higher enzyme activity of GLA, with even the lowest titer (i.e., 5E+10 vg/kg) resulting in higher levels of GLA enzyme activity than in control wild-type animals. In liver, the enzyme activity of GLA also increased with higher viral titers, with the lowest titer (i.e., 5E+10 vg/kg) resulting in comparable levels of enzyme activity of GLA with control wild-type animals. In kidney, the lowest and medium viral titers resulted in very low level of enzyme activity of GLA, but the highest titer (i.e., 5E+11 vg/kg) resulted in higher level of enzyme activity of GLA compared to the control wild-type animals. In heart, the level of enzyme activity of GLA increased with increased viral titers, with the medium viral titer of 1.5E+11 vg/kg resulting in comparable enzyme activity with control wild-type animals. Results show that when the virus dose is lower than 1.5E+11 vg/kg, the number of viruses reaching kidney through systemic administration is relatively few such that the level of enzyme activity of GLA expression in kidney upon administration of such a dose was lower than the expression level in wild type mice. Results are shown in FIG. 14A-FIG. 14D.

Renal involvement is a hallmark of Fabry disease and is mainly caused by globotriaosylceramide (Gb3) accumulation. Electron microscopy was used to visualize the ultrastructure of mouse renal parenchyma. In untreated Fabry model mice, the podocytes formed foot process fusion Gb3 accumulated, filtration slits formed multivesicular bodies and degraded, and the slit diaphragms formed a complex. These changes could potentially develop into proteinuria and glomerulosclerosis. It was observed that the lipid accumulation reduced in the entire renal parenchyma, which turned the renal structure back to normal (data not shown) in model mice with administration of ssAAV2/X-LP1-GLA at a high dose (i.e., 5E+11 vg/kg) substantially. The ultrastructure of kidneys of the treated model mice showed reduced number of lysosomes, reduced size of lysosomes, or less dense lysosomes, which demonstrates that the 5E+11 vg/kg rAAV dose level could both reduce Gb3 accumulation and prevent future Gb3 accumulation in model mice.

### Example 5.2: AAV2/X for Treatment of Hepatitis B

In Vitro Analysis of Suppression of HBV HBeAg, HBsAg, and HBV DNA by scAAV2/X-H1-shRNA-intron2 or scAAV2/8-H1-shRNA-intron2

scAAV2/X-H1-shRNA-intron2 and scAAV2/8-H1-shRNA-intron2 were used to infect HepG2.2.15 cells with increased Multiplicity of Infection (MOI). The levels of HBV HBeAg, HBV HBsAg, and HBV DNA in samples were measured by HBV HBeAg diagnostic kit (Beijing Wantai Biopharmaceuticals Ltd. Co.), HBV HBsAg diagnostic kit (Beijing Wantai Biopharmaceuticals Ltd. Co.), and HBV nucleotide quantification kit (QIAGEN), respectively. 1 ug/mL of Lamivudine (LAM) was used as a positive control in the experiments. scAAV2/X-H1-NC-intron2 (X-NC) and scAAV2/8-H1-NC-intron2 (8-NC) were used as negative controls. The NC sequence is a sequence that is unrelated to HBV and does not downregulate HBV when expressed as an shRNA. It is also unrelated to human genome and does not have RNA interference effects on human gene expression when expressed as an shRNA. The sample was collected every day for nine days and analyzed for the levels of HBeAg, HbsAg, and HBV DNA.

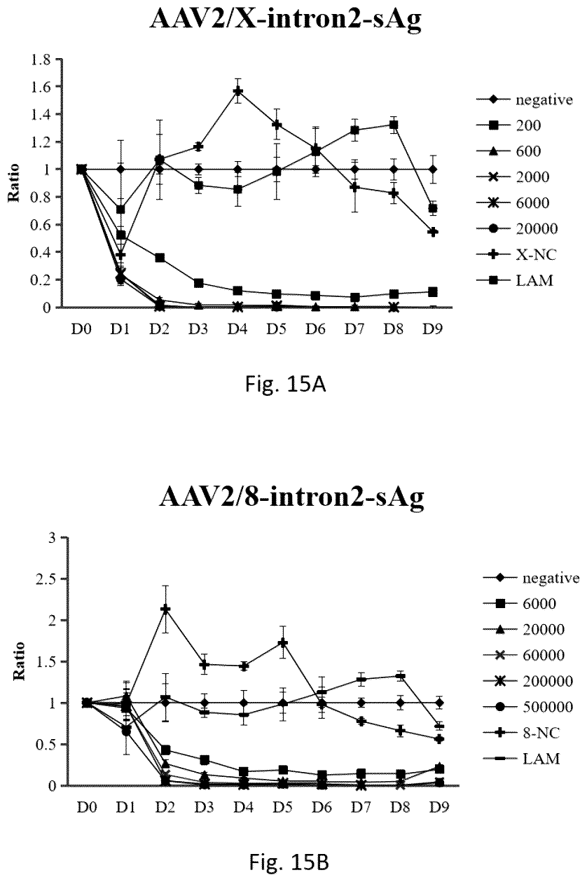

Results show that HBsAg levels started to decrease one day after infection and reached the lowest level on day 3. This low level was maintained until day 9. When the MOI of scAAV2/X-H1-shRNA-intron2 is greater than 6E+2, the greatest suppression of HBsAg levels was achieved. An MOI of scAAV2/X-H1-shRNA-intron2 of 6E+2 resulted in HBsAg levels approaching zero. When the MOI of scAAV2/8-H1-shRNA-intron2 is greater than 2E+4, the greatest suppression of HBsAg was achieved. An MOI of scAAV2/8-H1-shRNA-intron2 of 2E+4 resulted in HBsAg levels approaching zero. The results also show that when the two rAAVs achieved a similar inhibitory effect on HBsAg expression, the MOI of scAAV2/8-H1-shRNA-intron2 used was 30 times the MOI of scAAV2/X-H1-shRNA-intron2 used. By comparison, Lamivudine did not result in significant suppression of HBsAg. Results are shown in FIGS. 15A-15B and Tables 10 and 11.

TABLE 10
The level of HBsAg Detected in HepG2.2.15 Cells Infected with scAAV2/X-H1-shRNA-intron2

| MOI | D0 | D1 | D2 | D3 | D4 | D5 | D6 | D7 | D8 | D9 |
|---|---|---|---|---|---|---|---|---|---|---|
| Empty Virus | 1 | 1.0000002 | 0.9999999 | 0.9999999 | 1.0000001 | 1 | 0.9999999 | 1.0000001 | 1 | 1 |
| 2.00E+02 | 1 | 0.521621 | 0.3576298 | 0.1755682 | 0.1189527 | 0.0964428 | 0.0858919 | 0.0733121 | 0.0979604 | 0.1109952 |
| 6.00E+02 | 1 | 0.2366812 | 0.0523309 | 0.0157304 | 0.0130259 | 0.0153813 | 0.0034897 | 0.0048057 | 0.0020927 | −0.005433 |
| 2.00E+03 | 1 | 0.2427343 | 0.0142725 | −0.00363 | −0.012689 | 0.0078108 | −0.003822 | −0.001266 | −0.000645 | −0.044393 |
| 6.00E+03 | 1 | 0.2458644 | 0.0063115 | −0.0088 | 0.0016126 | 0.010292 | −0.002255 | −0.005722 | 0.0006226 | −0.063891 |
| 2.00E+04 | 1 | 0.2036345 | 0.0064395 | −0.001919 | −0.00706 | 0.0006203 | −0.002075 | −0.003616 | −0.003526 | −0.057936 |
| X-NC | 1 | 0.3831356 | 1.0730603 | 1.1635283 | 1.5671136 | 1.3258573 | 1.1517432 | 0.8699033 | 0.8283765 | 0.5483941 |
| Lamivudine | 1 | 0.7085067 | 1.0694985 | 0.8838135 | 0.8548879 | 0.9836865 | 1.1276321 | 1.284051 | 1.3238793 | 0.7174657 |

TABLE 11
The level of HBsAg Detected in HepG2.2.15 Cells Infected with SCAAV2/8-H1-shRNA-intron2

| MOI | D0 | D1 | D2 | D3 | D4 | D5 | D6 | D7 | D8 | D9 |
|---|---|---|---|---|---|---|---|---|---|---|
| Empty Virus | 1 | 1.0000003 | 1 | 0.9999998 | 1.0000001 | 1 | 0.9999999 | 0.9999999 | 1.0000001 | 1.0000004 |
| 6.00E+03 | 1 | 0.935851 | 0.4298982 | 0.3098707 | 0.1682881 | 0.1868237 | 0.1258725 | 0.1437872 | 0.1340821 | 0.195407 |
| 2.00E+04 | 1 | 1.0824304 | 0.2645846 | 0.1311427 | 0.0886794 | 0.0535988 | 0.043627 | 0.0389012 | 0.0492125 | 0.2314873 |
| 6.00E+04 | 1 | 0.9569798 | 0.1325721 | 0.036731 | 0.0254312 | 0.0175279 | 0.0062642 | 0.0031892 | 0.0036068 | 0.0440035 |
| 2.00E+05 | 1 | 0.960493 | 0.057368 | 0.0162515 | 0.011286 | 0.0273435 | 0.0185843 | 0.0008423 | −0.001134 | −0.014502 |
| 5.00E+05 | 1 | 0.6514033 | 0.0554953 | 0.0097407 | 0.0081587 | 0.0167903 | 0.0044997 | −0.001557 | −0.002518 | 0.0335568 |
| 8-NC | 1 | 0.9866094 | 2.130056 | 1.4639506 | 1.4446144 | 1.7294026 | 0.9775858 | 0.7784188 | 0.6623287 | 0.5614479 |
| Lamivudine | 1 | 0.7085067 | 1.0694985 | 0.8838135 | 0.8548879 | 0.9836865 | 1.1276321 | 1.284051 | 1.3238793 | 0.7174657 |

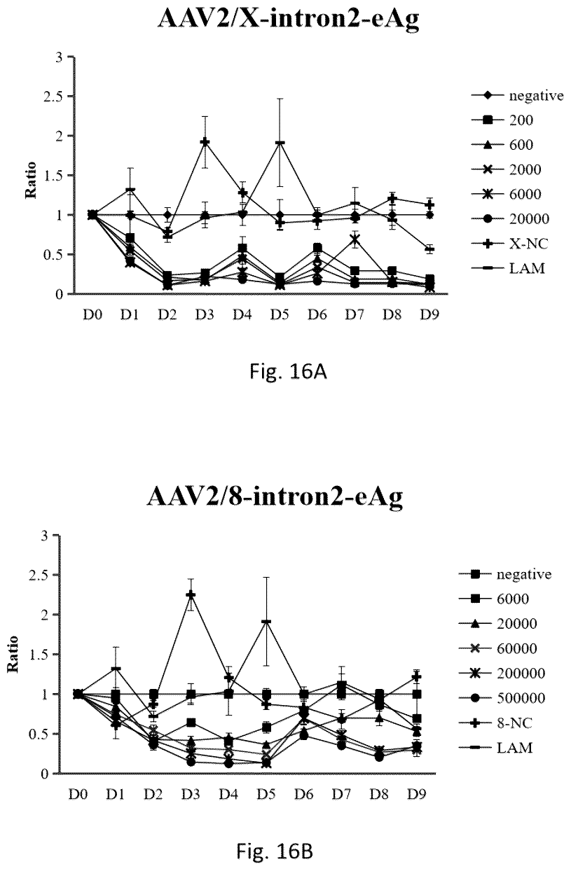

The levels of HBeAg also decreased one day after infection. Whereas scAAV2/X-H1-shRNA-intron2 achieved the strongest suppression of HBeAg on day 2, scAAV2/8-H1-shRNA-intron2 achieved the strongest suppression of HBeAg on day 3. HBeAg levels approached zero when the MOI of scAAV2/X-H1-shRNA-intron2 MOI was 2E+4 and was maintained at this low level from day 3 to day 9. HBeAg levels approached zero when the MOI of scAAV2/8-H1-shRNA-intron2 was 5E+5 and was maintained at this low level from day 4 to day 9. The results also show that when the two rAAV serotypes achieved a similar inhibitory effect on levels of HBsAg expression, the MOI of scAAV2/8-H1-shRNA-intron2 used was 25 times the MOI of scAAV2/X-H1-shRNA-intron2 used. By comparison, Lamivudine did not result in significant suppression of HBeAg. Results are shown in FIGS. 16A-16B and Tables 12 and 13.

TABLE 12
The level of HBeAg Detected in HepG2.2.15 Cells Infected with scAAV2/X-H1-shRNA-intron2

| MOI | D0 | D1 | D2 | D3 | D4 | D5 | D6 | D7 | D8 | D9 |
|---|---|---|---|---|---|---|---|---|---|---|
| Empty Virus | 1 | 1 | 1 | 1 | 1 | 1 | 1 | 1 | 1 | 1 |
| 2.00E+02 | 1 | 0.708415 | 0.238058 | 0.266593 | 0.57955 | 0.213115 | 0.577746 | 0.29089 | 0.292462 | 0.187972 |
| 6.00E+02 | 1 | 0.590939 | 0.211792 | 0.17377 | 0.481344 | 0.138781 | 0.448326 | 0.18771 | 0.1975 | 0.119562 |
| 2.00E+03 | 1 | 0.547653 | 0.166036 | 0.184826 | 0.443778 | 0.118746 | 0.337692 | 0.141368 | 0.148291 | 0.12779 |
| 6.00E+03 | 1 | 0.397325 | 0.109776 | 0.161181 | 0.274969 | 0.112709 | 0.259084 | 0.688945 | 0.150601 | 0.082363 |
| 2.00E+04 | 1 | 0.416802 | 0.107129 | 0.221975 | 0.180972 | 0.121418 | 0.161434 | 0.126361 | 0.129586 | 0.118465 |
| X-NC | 1 | 0.990185 | 0.786727 | 1.919656 | 1.284661 | 0.898263 | 0.924574 | 0.958366 | 1.207279 | 1.130845 |
| Lamivudine | 1 | 1.322533 | 0.715966 | 0.96118 | 1.034525 | 1.915765 | 0.997944 | 1.148177 | 0.936208 | 0.563239 |

TABLE 13
The level of HBeAg Detected in HepG2.2.15 Cells Infected with SCAAV2/8-H1-shRNA-intron2

| MOI | D0 | D1 | D2 | D3 | D4 | D5 | D6 | D7 | D8 | D9 |
|---|---|---|---|---|---|---|---|---|---|---|
| Empty Virus | 1 | 1 | 1 | 1.0000001 | 1.0000001 | 1.0000002 | 0.9999998 | 1.0000001 | 0.9999999 | 0.9999999 |
| 6.00E+03 | 1 | 0.9494641 | 0.4051664 | 0.6434651 | 0.4094359 | 0.5816686 | 0.7935469 | 1.1154798 | 0.8677254 | 0.6895081 |
| 2.00E+04 | 1 | 0.842982 | 0.4279938 | 0.4159068 | 0.4529442 | 0.3644979 | 0.5411713 | 0.6977691 | 0.7031804 | 0.5292912 |
| 6.00E+04 | 1 | 0.7417004 | 0.5446002 | 0.3174941 | 0.3003117 | 0.2389039 | 0.693772 | 0.4262907 | 0.266949 | 0.2942628 |
| 2.00E+05 | 1 | 0.6582259 | 0.4171171 | 0.2520517 | 0.1842672 | 0.1308529 | 0.6955081 | 0.4840955 | 0.289064 | 0.3336083 |
| 5.00E+05 | 1 | 0.7161095 | 0.3615699 | 0.1441351 | 0.1234292 | 0.1376073 | 0.4767613 | 0.3484958 | 0.2032049 | 0.3513105 |
| 8-NC | 1 | 0.6060134 | 0.8743558 | 2.2492778 | 1.2054464 | 0.8716624 | 0.8328438 | 0.6841938 | 0.9237801 | 1.2154254 |
| Lamivudine | 1 | 1.322533 | 0.7159665 | 0.9611801 | 1.0345255 | 1.9157645 | 0.9979437 | 1.1481768 | 0.936208 | 0.5632389 |

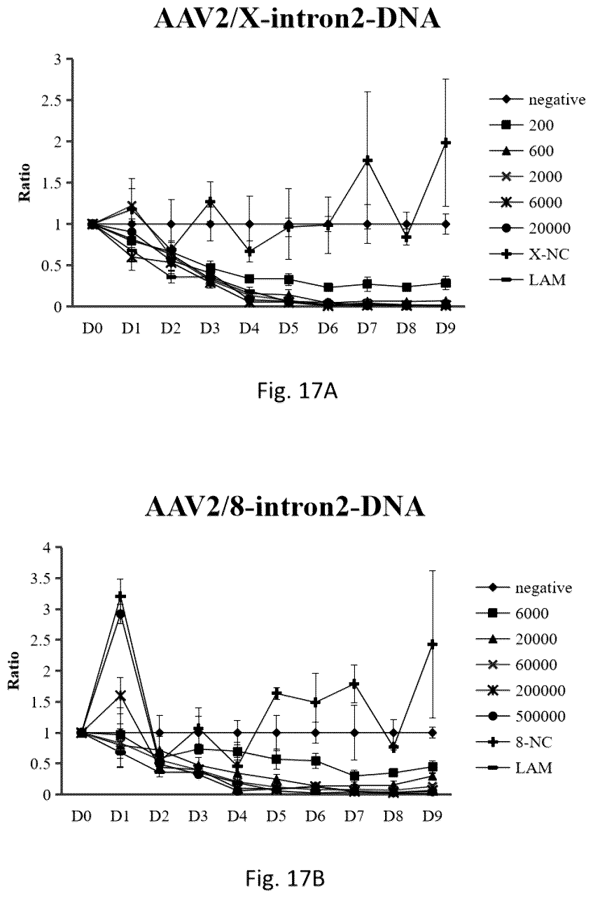

HBV DNA results show that DNA levels in cells infected by scAAV2/X-H1-shRNA-intron2 or scAAV2/8-H1-shRNA-intron2 started to decrease one day after infection and reached the lowest levels on day 5. The low HBV DNA levels in cells infected by either rAAV serotypes were maintained from day 6 to day 9. When the MOI of scAAV2/X-H1-shRNA-intron2 is greater than 2E+3, the lowest level of HBV DNA was observed. An MOI of scAAV2/X-H1-shRNA-intron2 of 2E+3 resulted in an HBV DNA level approaching zero. When the MOI of scAAV2/8-H1-shRNA-intron2 is greater than 2E+5, the lowest level of HBV DNA was observed. An MOI of scAAV2/8-H1-shRNA-intron2 of 2E+5 resulted in an HBV DNA level approaching zero. The results also show that when the two rAAV achieved a similar inhibitory effect on HBV DNA level, the MOI of scAAV2/8-H1-shRNA-intron2 used was 100 times the MOI of scAAV2/X-H1-shRNA-intron2 used. Results are shown in FIGS. 17A-17B and Tables 14 and 15.

TABLE 14
The level of HBV DNA Detected in HepG2.2.15 Cells Infected with SCAAV2/X-H1-shRNA-intron2

| MOI | D0 | D1 | D2 | D3 | D4 | D5 | D6 | D7 | D8 | D9 |
|---|---|---|---|---|---|---|---|---|---|---|
| Empty Virus | 1 | 1 | 1 | 1 | 1 | 1 | 1 | 1 | 1 | 1 |
| 2.00E+02 | 1 | 0.794513 | 0.663432 | 0.468956 | 0.3379 | 0.330579 | 0.231245 | 0.272403 | 0.239277 | 0.286585 |
| 6.00E+02 | 1 | 0.819469 | 0.631767 | 0.337065 | 0.158961 | 0.141736 | 0.044295 | 0.066288 | 0.060849 | 0.071196 |
| 2.00E+03 | 1 | 1.224779 | 0.609806 | 0.316933 | 0.134332 | 0.074008 | 0.019746 | 0.036691 | 0.014739 | 0.019588 |
| 6.00E+03 | 1 | 0.601239 | 0.533708 | 0.290499 | 0.054281 | 0.050785 | 0.009635 | 0.015888 | 0.0132 | 0.012375 |
| 2.00E+04 | 1 | 0.904425 | 0.556691 | 0.406397 | 0.082021 | 0.061901 | 0.049344 | 0.013058 | 0.013099 | 0.022201 |
| X-NC | 1 | 1.176235 | 0.685708 | 1.274876 | 0.670012 | 0.964083 | 0.989423 | 1.77193 | 0.844433 | 1.987109 |
| Lamivudine | 1 | 0.679706 | 0.358002 | 0.356965 | 0.19066 | 0.057353 | 0.023413 | 0.040011 | 0.021042 | 0.006153 |

TABLE 15
The level of HBV DNA Detected in HepG2.2.15 Cells Infected with SCAAV2/8-H1-shRNA-intron2

| MOI | D0 | D1 | D2 | D3 | D4 | D5 | D6 | D7 | D8 | D9 |
|---|---|---|---|---|---|---|---|---|---|---|
| Empty Virus | 1 | 1 | 1 | 1 | 1 | 1 | 1 | 1 | 1 | 1 |
| 6.00E+03 | 1 | 0.96669 | 0.588617 | 0.736503 | 0.701146 | 0.571008 | 0.546019 | 0.300387 | 0.349741 | 0.449937 |
| 2.00E+04 | 1 | 0.797307 | 0.717914 | 0.474617 | 0.345577 | 0.248471 | 0.139504 | 0.147744 | 0.156662 | 0.29975 |
| 6.00E+04 | 1 | 0.833451 | 0.559206 | 0.396374 | 0.205407 | 0.118913 | 0.077663 | 0.085174 | 0.067765 | 0.130038 |
| 2.00E+05 | 1 | 1.600283 | 0.440604 | 0.401289 | 0.101469 | 0.086546 | 0.129679 | 0.050967 | 0.032087 | 0.066583 |
| 5.00E+05 | 1 | 2.92275 | 0.493125 | 0.323529 | 0.060153 | 0.090849 | 0.126887 | 0.048088 | 0.03094 | 0.044869 |
| 8-NC | 1 | 3.205607 | 0.532377 | 1.074627 | 0.45851 | 1.635161 | 1.491346 | 1.791737 | 0.764108 | 2.43163 |
| Lamivudine | 1 | 0.679706 | 0.358002 | 0.356965 | 0.19066 | 0.057353 | 0.023413 | 0.040011 | 0.021042 | 0.006153 |

In Vivo Analysis of shRNA Efficacy Using Self-Complementary AAV2/8 and AAV2/X

scAAV2/X-H1-shRNA-intron2 and scAAV2/8-H1-shRNA-intron2 were administered to HBV transgenic mice (Beijing Vitalstar Biotechnology Co., Ltd, B6-Tg HBV/Vst; C57BL/6-HBV) via intravenous injection, respectively. Epivir® (Lamivudine) and Baraclude® (Entecavir) were used as controls. The experimental groups are shown in Table 16. Blood was collected on days 0, 7, 14, 21, 28, 35, 56, 84, 112, 140, 168, 196, 224, and 252, centrifuged, and assayed for the level of HBsAg, HBeAg, and HBV DNA.

TABLE 16
Administration of scAAV2/X-H1-shRNA-intron2 and
scAAV2/8-H1-shRNA-intron2 to HBV transgenic mice

| | Administered | | | Number of HBV |
|---|---|---|---|---|
| Group | Therapeutic Agent | Dose | | transgenic mice (n) |
| 1 | DPBS | 0.2 | mL/animal | 6 |
| 2 | Lamivudine | 150 | mg/kg | 6 |
| 3 | Entecavir | 3.2 | mg/kg | 6 |

| | | | |
|---|---|---|---|
| 4 | scAAV2/8-H1-shRNA-intron2 | 3 × 10¹⁰ vg/animal | 6 |
| 5 | scAAV2/X-H1-shRNA-intron2 | 3 × 10¹⁰ vg/animal | 6 |

Results show that the suppression of HBsAg was higher in animals administered with scAAV2/X-H1-shRNA-intron2 or scAAV2/8-H1-shRNA-intron2 than the controls (Table 17). Moreover, the reduction of HBsAg in animals administered with scAAV2/X-H1-shRNA-intron2 was 5-6 times greater than that in animals administered with scAAV2/8-H1-shRNA-intron2 (Table 17).

Further shown in Table 18, scAAV2/8-H1-shRNA-intron2 and Lamivudine had similar effects on HBV DNA levels, and the suppression of HBV DNA in animals administered with scAAV2/X-H1-shRNA-intron2 was 70 times greater than that in animals administered with scAAV2/8-H1-shRNA-intron2.

HBeAg levels were lower than 1 at most time points in animals administered with scAAV2/8-H1-shRNA-intron2, with more than 50% of animals not exhibiting detectable levels of HBeAg (statistical analysis was therefore not performed on these data). Administration of scAAV2/X-H1-shRNA-intron2 resulted in lowered HBeAg levels seven days post-injection. For example, at Day 56, administration of scAAV2/X-H1-shRNA-intron2 resulted in HBeAg reduction to 17.4±3.6 PEIU/mL (as shown in Table 19), whereas animals administered with DPBS exhibited no change in HBeAg levels (129.0 PEIU/mL compared to 133.9 PEIU/mL at D0). Administration of scAAV2/X-H1-shRNA-intron2 also resulted in lower HBsAg, HBeAg, and HBV DNA levels in the liver, with a 96%, 68%, and 91.6% decrease, respectively, compared to control at the last time point (Table 20).

TABLE 17
Comparison of HBsAg Levels Between Groups Administered with SCAAV2/8-H1-shRNA2-intron2 and scAAV2/X-H1-shRNA2-intron2

| Group | D0 | D7 | D14 | D21 | D28 | D35 | D56 | D84 | D112 | D140 | D168 | D196 | D224 | D252 |
|---|---|---|---|---|---|---|---|---|---|---|---|---|---|---|
| DPBS | 1 | 1.04 | 0.87 | 1.06 | 0.84 | 1.09 | 0.78 | 0.91 | 0.44 | 0.79 | 0.6 | 0.61 | 0.47 | 0.33 |
| LAM | 1 | 1 | 0.98 | 1.01 | 0.96 | 1.23 | 0.84 | 0.88 | 0.5 | 0.98 | 1.1 | 0.82 | 0.76 | 0.46 |
| ETV | 1 | 1.52 | 1.51 | 1.08 | 1.55 | 1.8 | 1.21 | 1.18 | 0.43 | 1.04 | 1.23 | 1.48 | 1.16 | 0.72 |
| SCAAV2/8-H1-shRNA2-intron2 | 1 | 0.1 | 0.04 | 0.01 | 0.07 | 0.09 | 0.08 | 0.07 | 0.04 | 0.13 | 0.12 | 0.13 | 0.1 | 0.06 |
| SCAAV2/X-H1-shRNA2-intron2 | 1 | 0.17 | 0.02 | 0.01 | 0.009 | 0.011 | 0.01 | 0.012 | 0.014 | 0.014 | 0.016 | 0.02 | 0.019 | 0.013 |

TABLE 18
Comparison of HBV DNA Levels Between Groups Administered with SCAAV2/8-H1-shRNA2-intron2 and
scAAV2/X-H1-shRNA2-intron2

| Group | D0 | D7 | D14 | D21 | D28 | D35 | D56 | D84 | D112 | D140 | D168 | D196 | D224 | D252 |
|---|---|---|---|---|---|---|---|---|---|---|---|---|---|---|
| DPBS | 1 | 3.96 | 1.13 | 0.85 | 2.85 | 2 | 1.28 | 1.39 | 6.92 | 1.87 | 1.74 | 3.58 | 3.03 | 6.66 |
| LAM | 1 | 1.3 | 0.69 | 0.49 | 0.87 | 0.72 | 0.97 | 0.43 | 1.64 | 0.92 | 0.63 | 0.18 | 0.12 | 0.23 |
| ETV | 1 | 5.7 | 8.68 | 0.66 | 1.24 | 1.08 | 1.17 | 1.1 | 2.24 | 1.91 | 1.79 | 2.17 | 2.48 | 1.42 |
| SCAAV2/8-H1-shRNA2-intron2 | 1 | 1.1 | 1.17 | 1.54 | 1.58 | 0.8 | 0.65 | 0.76 | 1.6 | 0.39 | 1.39 | 1.02 | 0.63 | 2.72 |
| SCAAV2/X-H1-shRNA2-intron2 | 1 | 0.29 | 0.038 | 0.04 | 0.013 | 0.018 | 0.017 | 0.022 | 0.025 | 0.01 | 0.012 | 0.012 | 0.01 | 0.01 |

TABLE 19
HBeAg Levels in Animals Administered with scAAV2/X-H1-shRNA2-intron2

| Group | D0 | D7 | D14 | D21 | D28 | D35 | D56 | D84 | D112 | D140 | D168 | D196 | D224 | D252 |
|---|---|---|---|---|---|---|---|---|---|---|---|---|---|---|
| DPBS | 133.9 | 135.6 | 89.3 | 89.9 | 71.3 | 50.5 | 129 | 103.5 | 135.3 | 172.3 | 120.7 | 155.6 | 123.6 | 137.9 |
| ETV | 130.7 | 138.6 | 92.1 | 66.5 | 78.3 | 48.2 | 131.4 | 105.1 | 133.8 | 178.5 | 125.9 | 150.5 | 123.5 | 155.1 |
| SCAAV2/ | 145.3 | 37.7 | 7.6 | 5.8 | 4.7 | 5.3 | 17.4 | 14.3 | 17 | 28.5 | 17.5 | 20.9 | 18.6 | 21.9 |
| X-H1-shRNA2-intron2 | | | | | | | | | | | | | | |

TABLE 20
HBsAg, HBeAg and HBV DNA Levels in
Liver Tissue at the Last Time Point

| | HBsAg | HBeAg | DNA |
|---|---|---|---|
| | (IU/g liver | (PEIU/g liver | (IU/g liver |
| Group | tissue) | tissue) | tissue) |
| DPBS | 7219.8 | 809.2 | 618683999 |
| scAAV2/X-H1- | 324.5 | 255.6 | 52118219 |
| shRNA2-intron2 | | | |

Sequences

SEQ ID NO: 1
MAADGYLPDWLEDNLSEGIREWWDLKPGAPKPKANQQKQDDGRGLVLPGYKYLG
PFNGLDKGEPVNAADAAALEHDKAYDQQLKAGDNPYLRYNHADAEFQERLQEDTS
FGGNLGRAVFQAKKRVLEPLGLVEEGAKTAPGKKRPVEQSPQEPDSSSGIGKTGQ
QPAKKRLNFGQTGDSESVPDPQPLGEPPAAPTSLGSNTMASGGGAPMADNNEGA
DGVGNASGNWHCDSTWLGDRVITTSTRTWALPTYNNHLYKQISSASTGASNDNHY
FGYSTPWGYFDFNRFHCHFSPRDWQRLINNNWGFRPKRLNFKLFNIQVKEVTTND
GVTTIANNLTSTVQVFSDSEYQLPYVLGSAHQGCLPPFPADVFMIPQYGYLTLNNG
SQAVGRSSFYCLEYFPSQMLRTGNNFTFSYTFEEVPFHSSYAHSQSLDRLMNPLID
QYLYYLNRTQNQSGSAQNKDLLFSRGSPAGMSVQPKNWLPGPCYRQQRVSKTKT
DNNNSNFTWTGASKYNLNGRESIINPGTAMASHKDDKDKFFPMSGVMIFGKESAG
ASNTALDNVMITDEEEIKATNPVATERFGTVAVNLQSSSTDPATGDVHVMGALPGM
VWQDRDVYLQGPIWAKIPHTDGHFHPSPLMGGFGLKHPPPQILIKNTPVPANPPAE
FSATKFASFITQYSTGQVSVEIEWELQKENSKRWNPEVQYTSNYAKSANVDFTVDN
NGLYTEPRPIGTRYLTRPL
SEQ ID NO: 2
MQLRNPELHLGCALALRFLALVSWDIPGARALDNGLARTPTMGWLHWERFMCNLD
CQEEPDSCISEKLFMEMAELMVSEGWKDAGYEYLCIDDCWMAPQRDSEGRLQAD
PQRFPHGIRQLANYVHSKGLKLGIYADVGNKTCAGFPGSFGYYDIDAQTFADWGV
DLLKFDGCYCDSLENLADGYKHMSLALNRTGRSIVYSCEWPLYMWPFQKPNYTEI
RQYCNHWRNFADIDDSWKSIKSILDWTSFNQERIVDVAGPGGWNDPDMLVIGNFG
LSWNQQVTQMALWAIMAAPLFMSNDLRHISPQAKALLQDKDVIAINQDPLGKQGYQ
LRQGDNFEVWERPLSGLAWAVAMINRQEIGGPRSYTIAVASLGKGVACNPACFITQ
LLPVKRKLGFYEWTSRLRSHINPTGTVLLQLENTMQMSLKDLL
SEQ ID NO: 3
gugugcacuucgcuucaccuucaagagaggugaagcgaagugcacac
SEQ ID NO: 4
gttcggctttacgtcacgcgagggcggcagggaggacggaatggcggggtttggggtgggtccctcctcgggggag
ccctgggaaaagaggactgcgtgtgggaagagaaggtggaaatggcgttttggttgacatgtgccgcctgcgagcg
tgctgcggggaggggccgagggcagattcgggaatgatggcgcggggtgggggcgtgggggctttctcgggaga
ggcccttccctggaagtttggggtgcgatggtgaggttctcggggcacctctggaggggcctcggcacggaaagcga
ccacctgggagggcgtgtggggaccaggttttgcctttagttttgcacacactgtagttcatctttatggagatgctcatgg
cctcattgaagccccactacagctctggtagcggtaaccatgcgtatttgacacacgaaggaactagggaaaaggc
attaggtcatttcaagccgaaattcacatgtgctagaatccagattccatgctgaccgatgccccaggatatagaaaat
gagaatctggtccttaccttcaagaacattcttaaccgtaatcagcctctggtatcttagctccaccctcactggttttttcttg
tttgttgaaccggccaagctgctggcctccctcctcaaccgttctgatcatgcttgctaaaatagtcaaaaccccggcca
gttaaatatgctttagcctgctttattatgattatttttgttgttttggcaatgacctggttacctgttgtttctcccactaaaacttttt
aagggcaggaatcaccgccgtaactctagcacttagcacagtacttggcttgtaagaggtcctcgatgatggtttgttg
aatgaatacattaaataattaaccacttgaaccctaagaaagaagcgattctatttcatattaggcattgtaatgacttaa
ggtaaagagcagtgctattaacggagtctaactgggaatccagcttgtttgggctatttactagttgtgtggctgtgggca
acttacttcacctctctgggcttaagtcattttatgtatatctgaggtgctggctacctcttggagttattgagaggattataag
acagtctatgtgaatcagcaacccttgcatggcccctggcggggaacagtaataatagccatcatcatgtttacttacat
agtcctaattagtcttcaaaacagccctgtagcaatggtatgattattaccattttacagatgaggaacctttgaagcctc
agagaggctaacagacataccctaggtcatacagttattaagagaaggagctctgtctcgaacctagctctctctctct
cgagtaataccagttaaaaaataggctacaaataggtactcaaaaaaatggtagtggctgttgtttttattcagttgctga
ggaaaaaatgttgatttttcatctctaaacatcaacttacttaattctgccaatttcttttttttgagacagggtctcactctgtc
acctaggatggagtgcagtggcacaatcactgctcactgcagcctcgacttcccgggctcgggtgattctccccaggc
tcaggggattctcccacttcagcctcccaagtagc
SEQ ID NO: 5
gaacgctgacgtcatcaacccgctccaaggaatcgcgggcccagtgtcactagggggaacacccagcgcgcgt
gcgccctggcaggaagatggctgtgagggacaggggagtggcgccctgcaatatttgcatgtcgctatgtgttctggg
aaatcaccataaacgtgaaatgtctttggatttgggaatcttataagttctgtatgagaccacagatctgtgtgcacttcgc
ttcaccttcaagagaggtgaagcgaagtgcacacttttttaagcttgttcggctttacgtcacgcgagggcggcaggga
ggacggaatggcggggtttggggtgggtccctcctcgggggagccctgggaaaagaggactgcgtgtgggaaga
gaaggtggaaatggcgttttggttgacatgtgccgcctgcgagcgtgctgcggggaggggccgagggcagattcgg
gaatgatggcgcggggtgggggcgtgggggctttctcgggagaggcccttccctggaagtttggggtgcgatggtga
ggttctcggggcacctctggaggggcctcggcacggaaagcgaccacctgggagggcgtgtggggaccaggttttg
cctttagttttgcacacactgtagttcatctttatggagatgctcatggcctcattgaagccccactacagctctggtagcg
gtaaccatgcgtatttgacacacgaaggaactagggaaaaggcattaggtcatttcaagccgaaattcacatgtgcta
gaatccagattccatgctgaccgatgccccaggatatagaaaatgagaatctggtccttaccttcaagaacattcttaa
ccgtaatcagcctctggtatcttagctccaccctcactggttttttcttgtttgttgaaccggccaagctgctggcctccctcct
caaccgttctgatcatgcttgctaaaatagtcaaaaccccggccagttaaatatgctttagcctgctttattatgattattttt
gttgttttggcaatgacctggttacctgttgtttctcccactaaaactttttaagggcaggaatcaccgccgtaactctagca
cttagcacagtacttggcttgtaagaggtcctcgatgatggtttgttgaatgaatacattaaataattaaccacttgaaccc
taagaaagaagcgattctatttcatattaggcattgtaatgacttaaggtaaagagcagtgctattaacggagtctaact
gggaatccagcttgtttgggctatttactagttgtgtggctgtgggcaacttacttcacctctctgggcttaagtcattttatgt
atatctgaggtgctggctacctcttggagttattgagaggattataagacagtctatgtgaatcagcaacccttgcatggc
ccctggcggggaacagtaataatagccatcatcatgtttacttacatagtcctaattagtcttcaaaacagccctgtagc
aatggtatgattattaccattttacagatgaggaacctttgaagcctcagagaggctaacagacataccctaggtcata
cagttattaagagaaggagctctgtctcgaacctagctctctctctctcgagtaataccagttaaaaaataggctacaa
ataggtactcaaaaaaatggtagtggctgttgtttttattcagttgctgaggaaaaaatgttgatttttcatctctaaacatca
acttacttaattctgccaatttcttttttttgagacagggtctcactctgtcacctaggatggagtgcagtggcacaatcactg
ctcactgcagcctcgacttcccgggctcgggtgattctccccaggctcaggggattctcccacttcagcctcccaagta
gc
SEQ ID NO: 6
gaattggagatcggtacttcgcgaatgcgtcgagttaatttttaaaaagcagtcaaaagtccaagtggcccttggcagc
atttactctctctgtttgctctggttaataatctcaggagcacaaacattcctggaggcaggagaagaaatcaacatcct
ggacttatcctctgggcctctccccacccccaggagaggctgtgcaactgttaatttttaaaaagcagtcaaaagtcca
agtggcccttggcagcatttactctctctgtttgctctggttaataatctcaggagcacaaacattcctggaggcaggaga
agaaatcaacatcctggacttatcctctgggcctctccccacccccaggagaggctgtgcaactggatccaggcctg
aggctggtcaaaattgaacctcctcctgctctgagcagcctggggggcagactaagcagagggctgtgcagaccca
cataaagagcctactgtgtgccaggcacttcacccgaggcacttcacaagcatgcttgggaatgaaacttccaactct
ttgggatgcaggtgaaacagttcctggttcagagaggtgaagcggcctgcctgaggcagcacagctcttctttacaga
tgtgcttccccacctctaccctgtctcacggccccccatgccagcctgacggttgtgtctgcctcagtcatgctccatttttc
catcgggaccatcaagagggtgtttgtgtctaaggctgactgggtaactttggatgagcggtctctccgctctgagcctg
tttcctcatctgtcaaatgggctctaacccactctgatctcccagggcggcagtaagtcttcagcatcaggcattttgggg
tgactcagtaaatggtagatcttgctaccagtggaacagccactaaggattctgcagtgagagcagagggccagcta
agtggtactctcccagagactgtctgactcacgccaccccctccaccttggacacaggacgctgtggtttctgagcca
ggtacaatgactcctttcggtaagtgcagtggaagctgtacactgcccaggcaaagcgtccgggcagcgtaggcgg
gcgactcagatcccagccagtggacttagcccctgtttgctcctccgataactggggtgaccttggttaatattcaccag
cagcctcccccgttgcccctctggatccactgcttaaatacggacgaggacagggccctgtctcctcagcttcaggca
ccaccactgacctgggacagtgaatcgcggccgcatatgccaccatgcagctgaggaacccagaactacatctgg
gctgcgcgcttgcgcttcgcttcctggccctcgtttcctgggacatccctggggctagagcactggacaatggattggca
aggacgcctaccatgggctggctgcactgggagcgcttcatgtgcaaccttgactgccaggaagagccagattcctg
catcagtgagaagctcttcatggagatggcagagctcatggtctcagaaggctggaaggatgcaggttatgagtacct
ctgcattgatgactgttggatggctccccaaagagattcagaaggcagacttcaggcagaccctcagcgctttcctcat
gggattcgccagctagctaattatgttcacagcaaaggactgaagctagggatttatgcagatgttggaaataaaacc
tgcgcaggcttccctgggagttttggatactacgacattgatgcccagacctttgctgactggggagtagatctgctaaa
atttgatggttgttactgtgacagtttggaaaatttggcagatggttataagcacatgtccttggccctgaataggactggc
agaagcattgtgtactcctgtgagtggcctctttatatgtggccctttcaaaagcccaattatacagaaatccgacagtac
tgcaatcactggcgaaattttgctgacattgatgattcctggaaaagtataaagagtatcttggactggacatcttttaacc
aggagagaattgttgatgttgctggaccagggggttggaatgacccagatatgttagtgattggcaactttggcctcag
ctggaatcagcaagtaactcagatggccctctgggctatcatggctgctcctttattcatgtctaatgacctccgacacat
cagccctcaagccaaagctctccttcaggataaggacgtaattgccatcaatcaggaccccttgggcaagcaaggg
taccagcttagacagggagacaactttgaagtgtgggaacgacctctctcaggcttagcctgggctgtagctatgata
aaccggcaggagattggtggacctcgctcttataccatcgcagttgcttccctgggtaaaggagtggcctgtaatcctg
cctgcttcatcacacagctcctccctgtgaaaaggaagctagggttctatgaatggacttcaaggttaagaagtcacat
aaatcccacaggcactgttttgcttcagctagaaaatacaatgcagatgtcattaaaagacttactttaa
SEQ ID NO: 7
ccctaaaatgggcaaacattgcaagcagcaaacagcaaacacacagccctccctgcctgctgaccttggagctgg
ggcagaggtcagagacctctctgggcccatgccacctccaacatccactcgaccccttggaatttcggtggagagga
gcagaggttgtcctggcgtggtttaggtagtgtgagaggggaatgactcctttcggtaagtgcagtggaagctgtacac
tgcccaggcaaagcgtccgggcagcgtaggcgggcgactcagatcccagccagtggacttagcccctgtttgctcct
ccgataactggggtgaccttggttaatattcaccagcagcctcccccgttgcccctctggatccactgcttaaatacgga
cgaggacagggccctgtctcctcagcttcaggcaccaccactgacctgggacagtgaatccggactctaaggtaaat
ataaaatttttaagtgtataatgtgttaaactactgattctaattgtttctctcttttagattccaacctttggaactgaattctag
accaccgcggccgcatatgccaccatgcagctgaggaacccagaactacatctgggctgcgcgcttgcgcttcgctt
cctggccctcgtttcctgggacatccctggggctagagcactggacaatggattggcaaggacgcctaccatgggct
ggctgcactgggagcgcttcatgtgcaaccttgactgccaggaagagccagattcctgcatcagtgagaagctcttca
tggagatggcagagctcatggtctcagaaggctggaaggatgcaggttatgagtacctctgcattgatgactgttggat
ggctccccaaagagattcagaaggcagacttcaggcagaccctcagcgctttcctcatgggattcgccagctagcta
attatgttcacagcaaaggactgaagctagggatttatgcagatgttggaaataaaacctgcgcaggcttccctggga
gttttggatactacgacattgatgcccagacctttgctgactggggagtagatctgctaaaatttgatggttgttactgtgac
agtttggaaaatttggcagatggttataagcacatgtccttggccctgaataggactggcagaagcattgtgtactcctgt
gagtggcctctttatatgtggccctttcaaaagcccaattatacagaaatccgacagtactgcaatcactggcgaaatttt
gctgacattgatgattcctggaaaagtataaagagtatcttggactggacatcttttaaccaggagagaattgttgatgtt
gctggaccagggggttggaatgacccagatatgttagtgattggcaactttggcctcagctggaatcagcaagtaact
cagatggccctctgggctatcatggctgctcctttattcatgtctaatgacctccgacacatcagccctcaagccaaagc
tctccttcaggataaggacgtaattgccatcaatcaggaccccttgggcaagcaagggtaccagcttagacagggag
acaactttgaagtgtgggaacgacctctctcaggcttagcctgggctgtagctatgataaaccggcaggagattggtg
gacctcgctcttataccatcgcagttgcttccctgggtaaaggagtggcctgtaatcctgcctgcttcatcacacagctcc
tccctgtgaaaaggaagctagggttctatgaatggacttcaaggttaagaagtcacataaatcccacaggcactgtttt
gcttcagctagaaaatacaatgcagatgtcattaaaagacttactttaa
SEQ ID NO: 8
gtcgacaccggttagtaatgatcgacaatcaacctctggattacaaaatttgtgaaagattgactggtattcttaactatgt
tgctccttttacgctatgtggatacgctgctttaatgcctttgtatcatgctattgcttcccgtatggctttcattttctcctccttgt
ataaatcctggttgctgtctctttatgaggagttgtggcccgttgtcaggcaacgtggcgtggtgtgcactgtgtttgctgac
gcaacccccactggttggggcattgccaccacctgtcagctcctttccgggactttcgctttccccctccctattgccacg
gcggaactcatcgccgcctgccttgcccgctgctggacaggggctcggctgttgggcactgacaattccgtggtgttgt
cggggaagctgacgtcctttccatggctgctcgcctgtgttgccacctggattctgcgcgggacgtccttctgctacgtcc
cttcggccctcaatccagcggaccttccttcccgcggcctgctgccggctctgcggcctcttccgcgtcttcgccttcgcc
ctcagacgagtcggatctccctttgggccgcctc
SEQ ID NO: 9
Gaacgctgacgtcatcaacccgctccaaggaatcgcgggcccagtgtcactaggcgggaacacccagcgcgcgt
gcgccctggcaggaagatggctgtgagggacaggggagtggcgccctgcaatatttgcatgtcgctatgtgttctggg
aaatcaccataaacgtgaaatgtctttggatttgggaatcttataagttctgtatgagaccac
SEQ ID NO: 10
atggctgccgatggttatcttccagattggctcgaggacaacctctctgagggcattcgcgagtggtgggacttgaaac
ctggagccccgaagcccaaagccaaccagcaaaagcaggacgacggccggggtctggtgcttcctggctacaag
tacctcggacccttcaacggactcgacaagggggagcccgtcaacgcggcggacgcagcggccctcgagcacg
acaaggcctacgaccagcagctcaaagcgggtgacaatccgtacctgcggtataaccacgccgacgccgagtttc
aggagcgtctgcaagaagatacgtcttttgggggcaacctcgggcgagcagtcttccaggccaagaagcgggttctc
gaacctctcggtctggttgaggaaggcgctaagacggctcctggaaagaaacgtccggtagagcagtcgccacaa
gagccagactcctcctcgggcatcggcaagacaggccagcagcccgctaaaaagagactcaattttggtcagact
ggcgactcagagtcagttccagaccctcaacctctcggagaaccaccagcagcccccacaagtttgggatctaata
caatggcttcaggcggtggcgcaccaatggcagacaataacgaaggcgccgacggagtgggtaatgcctcagga
aattggcattgcgattccacatggctgggcgacagagtcatcaccaccagcacccgaacatgggccttgcccaccta
taacaaccacctctacaagcaaatctccagtgcttcaacgggggccagcaacgacaaccactacttcggctacagc
accccctgggggtattttgatttcaacagattccactgccatttctcaccacgtgactggcagcgactcatcaacaacaa
ttggggattccggcccaagagactcaacttcaagctcttcaacatccaagtcaaggaggtcacgacgaatgatggcg
tcacgaccatcgctaataaccttaccagcacggttcaagtcttctcggactcggagtaccagttgccgtacgtcctcgg
ctctgcgcaccagggctgcctccctccgttcccggcggacgtgttcatgattccgcaatacggctacctgacgctcaac
aatggcagccaagccgtgggacgttcatccttttactgcctggaatatttcccttctcagatgctgagaacgggcaaca
actttaccttcagctacacctttgaggaagtgcctttccacagcagctacgcgcacagccagagcctggaccggctga
tgaatcctctcatcgaccagtacctgtattacctgaacagaactcagaatcagtccggaagtgcccaaaacaaggac
ttgctgtttagccgtgggtctccagctggcatgtctgttcagcccaaaaactggctacctggaccctgttaccggcagca
gcgcgtttctaaaacaaaaacagacaacaacaacagcaactttacctggactggtgcttcaaaatataacctcaatg
ggcgtgaatccatcatcaaccctggcactgctatggcctcacacaaagacgacaaagacaagttctttcccatgagc
ggtgtcatgatttttggaaaggagagcgccggagcttcaaacactgcattggacaatgtcatgatcacagacgaaga
ggaaatcaaagccactaaccccgtggccaccgaaagatttgggactgtggcagtcaatctccagagcagcagcac
agaccctgcgaccggagatgtgcatgttatgggagccttacctggaatggtgtggcaagacagagacgtatacctgc
agggtcctatttgggccaaaattcctcacacggatggacactttcacccgtctcctctcatgggcggctttggacttaagc
acccgcctcctcagatcctcatcaaaaacacgcctgttcctgcgaatcctccggcagagttttcggctacaaagtttgct
tcattcatcacccagtattccacaggacaagtgagcgtggagattgaatgggagctgcagaaagaaaacagcaaa
cgctggaatcccgaagtgcagtatacatctaactatgcaaaatctgccaacgttgattttactgtggacaacaatggac
tttatactgagcctcgccccattggcacccgttaccttacccgtcccctgtaa
SEQ ID NO: 20
gaattggagatcggtacttcgcgaatgcgtcgagttaatttttaaaaagcagtcaaaagtccaagtggcccttggcagc
atttactctctctgtttgctctggttaataatctcaggagcacaaacattcctggaggcaggagaagaaatcaacatcct
ggacttatcctctgggcctctccccacccccaggagaggctgtgcaactgttaatttttaaaaagcagtcaaaagtcca
agtggcccttggcagcatttactctctctgtttgctctggttaataatctcaggagcacaaacattcctggaggcaggaga
agaaatcaacatcctggacttatcctctgggcctctccccacccccaggagaggctgtgcaactggatccaggcctg
aggctggtcaaaattgaacctcctcctgctctgagcagcctggggggcagactaagcagagggctgtgcagaccca
cataaagagcctactgtgtgccaggcacttcacccgaggcacttcacaagcatgcttgggaatgaaacttccaactct
ttgggatgcaggtgaaacagttcctggttcagagaggtgaagcggcctgcctgaggcagcacagctcttctttacaga
tgtgcttccccacctctaccctgtctcacggccccccatgccagcctgacggttgtgtctgcctcagtcatgctccatttttc
catcgggaccatcaagagggtgtttgtgtctaaggctgactgggtaactttggatgagcggtctctccgctctgagcctg
tttcctcatctgtcaaatgggctctaacccactctgatctcccagggcggcagtaagtcttcagcatcaggcattttgggg
tgactcagtaaatggtagatcttgctaccagtggaacagccactaaggattctgcagtgagagcagagggccagcta
agtggtactctcccagagactgtctgactcacgccaccccctccaccttggacacaggacgctgtggtttctgagcca
ggtacaatgactcctttcggtaagtgcagtggaagctgtacactgcccaggcaaagcgtccgggcagcgtaggcgg
gcgactcagatcccagccagtggacttagcccctgtttgctcctccgataactggggtgaccttggttaatattcaccag
cagcctcccccgttgcccctctggatccactgcttaaatacggacgaggacagggccctgtctcctcagcttcaggca
ccaccactgacctgggacagtgaatc
SEQ ID NO: 21
Ccctaaaatgggcaaacattgcaagcagcaaacagcaaacacacagccctccctgcctgctgaccttggagctgg
ggcagaggtcagagacctctctgggcccatgccacctccaacatccactcgaccccttggaatttcggtggagagga
gcagaggttgtcctggcgtggtttaggtagtgtgagaggggaatgactcctttcggtaagtgcagtggaagctgtacac
tgcccaggcaaagcgtccgggcagcgtaggcgggcgactcagatcccagccagtggacttagcccctgtttgctcct
ccgataactggggtgaccttggttaatattcaccagcagcctcccccgttgcccctctggatccactgcttaaatacgga
cgaggacagggccctgtctcctcagcttcaggcaccaccactgacctgggacagtgaatccggactctaaggtaaat
ataaaatttttaagtgtataatgtgttaaactactgattctaattgtttctctcttttagattccaacctttggaactg
SEQ ID NO: 22
gtacctcgcgaatgcatctacgatgcgagaacttgtgcctccccgtgttcctgctctttgtccctctgtcctacttagactaa
tatttgccttgggtactgcaaacaggaaatgggggagggattcgaaacggctttgcggacactagcgatgcgagaac
ttgtgcctccccgtgttcctgctctttgtccctctgtcctacttagactaatatttgccttgggtactgcaaacaggaaatggg
ggagggattcgaaacggctttgcggacactagtgtgtgcatgcgtgagtacttgtgtgtaaatttttcattatctataggta
aaagcacacttggaattagcaatagatgcaatttgggacttaactctttcagtatgtcttatttctaagcaaagtatttagttt
ggttagtaattactaaacactgagaactaaattgcaaacaccaagaactaaaatgttcaagtgggaaattacagttaa
ataccatggtaatgaataaaaggtacaaatcgtttaaactcttatgtaaaatttgataagatgttttacacaactttaatac
attgacaaggtcttgtggagaaaacagttccagatggtaaatatacacaagggatttagtcaaacaattttttggcaag
aatattatgaattttgtaatcggttggcagccaatgaaatacaaagatgagtctagttaataatctacaattattggttaaa
gaagtatattagtgctaatttccctccgtttgtcctagcttttctcttctgtcaac

1. A recombinant adeno-associated virus (rAAV) comprising:
(a) an adeno-associated virus (AAV) capsid protein having the amino acid sequence of SEQ ID NO: 1; and
(b) an expression cassette comprising a polynucleotide sequence, wherein the polynucleotide sequence encodes a therapeutic agent useful in gene therapy treatment of a liver disease.

2. The rAAV of claim 1, wherein the therapeutic agent encoded by the polynucleotide sequence is alpha galactosidase A (GLA) or an shRNA targeting a Hepatitis B virus (HBV) genome.

3. The rAAV of claim 1, wherein the expression cassette further comprises a promoter and/or a human non-encoding filler sequence, wherein the promoter is located upstream of the polynucleotide sequence and the human non-encoding filler sequence is located downstream of the polynucleotide sequence.

4. The rAAV of claim 3, wherein the promoter is an RNA polymerase II promoter or an RNA polymerase III promoter.

5. The rAAV of claim 3, wherein the promoter is an LP1 promoter, an ApoE/hAAT promoter, a DC172 promoter, a DC190 promoter, an ApoA-I promoter, a TBG promoter, an LSP1 promoter, a 7SK promoter, an H1 promoter, a U6 promoter, or an HD-IFN promoter.

6. The rAAV of claim 3, wherein the promoter is:
(i) an LP1 or DC172 promoter for the polynucleotide sequence encoding the GLA, or
(ii) an H1 promoter for the polynucleotide sequence encoding the shRNA.

7. The rAAV of claim 1, wherein the expression cassette further comprises a first AAV inverted terminal repeat (ITR) and a second AAV ITR.

8. The rAAV of claim 7, wherein the first and/or second ITR are/is derived from a serotype of AAV in clades A-F, AAV1, AAV2, AAV3, AAV4, AAV5, AAV6, AAV7, AAV8, AAV9, or any hybrid/chimeric types thereof.

9. The rAAV of claim 7, wherein the first and/or second ITR are/is derived from an AAV2 serotype.

10. The rAAV of claim 2, wherein:
(i) the GLA comprises SEQ ID NO: 2, or
(ii) the polynucleotide sequence encoding the shRNA comprises SEQ ID NO: 3.

11. The rAAV of claim 3, wherein the human non-encoding filler sequence is an intron sequence of human factor IX, a sequence of human cosmid C346, an HPRT-intron sequence, or combinations thereof.

12. The rAAV of claim 11, wherein the human non-encoding filler sequence is an HPRT-intron sequence comprising SEQ ID NO: 4.

13. The rAAV of claim 1, wherein the expression cassette comprises SEQ ID NO: 5.

14. The rAAV of claim 1, wherein the expression cassette comprises SEQ ID NO: 6 or 7.

15. The rAAV of claim 14, wherein the expression cassette further comprises SEQ ID NO: 8.

16. A composition comprising the rAAV of claim 1 and a pharmaceutically acceptable excipient and/or diluent.

17. (canceled)

18. A method of treating a liver disease in a patient in need thereof, comprising administering to the patient a therapeutically effective amount of the rAAV of claim 1.

19. The method of claim 18, wherein the liver disease is Fabry disease or Hepatitis B.

20. The method of claim 18, wherein the rAAV or the composition is administered intravenously.

21. The method of claim 18, further comprising administering a second therapeutic agent.

22. The method of claim 18, wherein administration of the rAAV or the composition results in:
(i) an increased level of GLA expression in a liver tissue compared to a corresponding rAAV comprising an AAV2/8 serotype capsid protein; or
(ii) an increased inhibition of Hepatitis B surface antigen (HBsAg), Hepatitis B e-antigen (HBeAg), or HBV DNA, compared to a corresponding rAAV comprising an AAV2/8 serotype capsid protein.

23. The method of claim 18, wherein the therapeutically effective amount of the rAAV comprises about 1×10⁶ VG to about 1×10¹⁸ VG.

24. (canceled)

25. (canceled)

26. (canceled)

27. (canceled)

28. (canceled)

29. (canceled)

30. (canceled)

31. (canceled)

32. (canceled)

33. (canceled)


SEQUENCE LISTING (22 sequences)

SEQ ID NO: 1 - protein, 736 aa, Artificial Sequence
  Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPTSLGSNTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 3 - RNA, 47 nt, Artificial Sequence
  Synthetic polynucleotide
gugugcacuucgcuucaccuucaagagaggugaagcgaagugcacac

SEQ ID NO: 4 - DNA, 1642 bp, Artificial Sequence
  Synthetic polynucleotide
gttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccactacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggttacctgttgtttctcccactaaaactttttaagggcaggaatcaccgccgtaactctagcacttagcacagtacttggcttgtaagaggtcctcgatgatggtttgttgaatgaatacattaaataattaaccacttgaaccctaagaaagaagcgattctatttcatattaggcattgtaatgacttaaggtaaagagcagtgctattaacggagtctaactgggaatccagcttgtttgggctatttactagttgtgtggctgtgggcaacttacttcacctctctgggcttaagtcattttatgtatatctgaggtgctggctacctcttggagttattgagaggattataagacagtctatgtgaatcagcaacccttgcatggcccctggcggggaacagtaataatagccatcatcatgtttacttacatagtcctaattagtcttcaaaacagccctgtagcaatggtatgattattaccattttacagatgaggaacctttgaagcctcagagaggctaacagacataccctaggtcatacagttattaagagaaggagctctgtctcgaacctagctctctctctctcgagtaataccagttaaaaaataggctacaaataggtactcaaaaaaatggtagtggctgttgtttttattcagttgctgaggaaaaaatgttgatttttcatctctaaacatcaacttacttaattctgccaatttcttttttttgagacagggtctcactctgtcacctaggatggagtgcagtggcacaatcactgctcactgcagcctcgacttcccgggctcgggtgattctccccaggctcaggggattctcccacttcagcctcccaagtagc

SEQ ID NO: 5 - DNA, 1922 bp, Artificial Sequence
  Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctgtgtgcacttcgcttcaccttcaagagaggtgaagcgaagtgcacacttttttaagcttgttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccactacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggttacctgttgtttctcccactaaaactttttaagggcaggaatcaccgccgtaactctagcacttagcacagtacttggcttgtaagaggtcctcgatgatggtttgttgaatgaatacattaaataattaaccacttgaaccctaagaaagaagcgattctatttcatattaggcattgtaatgacttaaggtaaagagcagtgctattaacggagtctaactgggaatccagcttgtttgggctatttactagttgtgtggctgtgggcaacttacttcacctctctgggcttaagtcattttatgtatatctgaggtgctggctacctcttggagttattgagaggattataagacagtctatgtgaatcagcaacccttgcatggcccctggcggggaacagtaataatagccatcatcatgtttacttacatagtcctaattagtcttcaaaacagccctgtagcaatggtatgattattaccattttacagatgaggaacctttgaagcctcagagaggctaacagacataccctaggtcatacagttattaagagaaggagctctgtctcgaacctagctctctctctctcgagtaataccagttaaaaaataggctacaaataggtactcaaaaaaatggtagtggctgttgtttttattcagttgctgaggaaaaaatgttgatttttcatctctaaacatcaacttacttaattctgccaatttcttttttttgagacagggtctcactctgtcacctaggatggagtgcagtggcacaatcactgctcactgcagcctcgacttcccgggctcgggtgattctccccaggctcaggggattctcccacttcagcctcccaagtagc

SEQ ID NO: 6 - DNA, 2595 bp, Artificial Sequence
  Synthetic polynucleotide
gaattggagatcggtacttcgcgaatgcgtcgagttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggagaggctgtgcaactgttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggagaggctgtgcaactggatccaggcctgaggctggtcaaaattgaacctcctcctgctctgagcagcctggggggcagactaagcagagggctgtgcagacccacataaagagcctactgtgtgccaggcacttcacccgaggcacttcacaagcatgcttgggaatgaaacttccaactctttgggatgcaggtgaaacagttcctggttcagagaggtgaagcggcctgcctgaggcagcacagctcttctttacagatgtgcttccccacctctaccctgtctcacggccccccatgccagcctgacggttgtgtctgcctcagtcatgctccatttttccatcgggaccatcaagagggtgtttgtgtctaaggctgactgggtaactttggatgagcggtctctccgctctgagcctgtttcctcatctgtcaaatgggctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcggccgcatatgccaccatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 7 - DNA, 1866 bp, Artificial Sequence
  Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccaccgcggccgcatatgccaccatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 8 - DNA, 610 bp, Artificial Sequence
  Synthetic polynucleotide
gtcgacaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctc

SEQ ID NO: 9 - DNA, 215 bp, Artificial Sequence
  Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 10 - DNA, 2211 bp, Artificial Sequence
  Synthetic polynucleotide
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagttccagaccctcaacctctcggagaaccaccagcagcccccacaagtttgggatctaatacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccagtacctgtattacctgaacagaactcagaatcagtccggaagtgcccaaaacaaggacttgctgtttagccgtgggtctccagctggcatgtctgttcagcccaaaaactggctacctggaccctgttaccggcagcagcgcgtttctaaaacaaaaacagacaacaacaacagcaactttacctggactggtgcttcaaaatataacctcaatgggcgtgaatccatcatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgattttactgtggacaacaatggactttatactgagcctcgccccattggcacccgttaccttacccgtcccctgtaa

SEQ ID NO: 11 - DNA, 23 bp, Artificial Sequence
  Synthetic polynucleotide
cgagaacaacgaagacttcaaca

SEQ ID NO: 12 - DNA, 18 bp, Artificial Sequence
  Synthetic polynucleotide
cgcggtcagcatcgagat

SEQ ID NO: 13 - DNA, 20 bp, Artificial Sequence
  Synthetic polynucleotide
ccgtggccagcaacttcgcg

SEQ ID NO: 14 - DNA, 20 bp, Artificial Sequence
  Synthetic polynucleotide
ctgccaggaagagccagatt

SEQ ID NO: 15 - DNA, 24 bp, Artificial Sequence
  Synthetic polynucleotide
gtactcataacctgcatccttcca

SEQ ID NO: 16 - DNA, 16 bp, Artificial Sequence
  Synthetic polynucleotide
tgcatcagtgagaagc

SEQ ID NO: 17 - DNA, 20 bp, Artificial Sequence
  Synthetic polynucleotide
atcaacccgctccaaggaat

SEQ ID NO: 18 - DNA, 25 bp, Artificial Sequence
  Synthetic polynucleotide
aacacatagcgacatgcaaatattg

SEQ ID NO: 19 - DNA, 25 bp, Artificial Sequence
  Synthetic polynucleotide
cccagtgtcactaggcgggaacacc

SEQ ID NO: 20 - DNA, 1287 bp, Artificial Sequence
  Synthetic polynucleotide
gaattggagatcggtacttcgcgaatgcgtcgagttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggagaggctgtgcaactgttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggagaggctgtgcaactggatccaggcctgaggctggtcaaaattgaacctcctcctgctctgagcagcctggggggcagactaagcagagggctgtgcagacccacataaagagcctactgtgtgccaggcacttcacccgaggcacttcacaagcatgcttgggaatgaaacttccaactctttgggatgcaggtgaaacagttcctggttcagagaggtgaagcggcctgcctgaggcagcacagctcttctttacagatgtgcttccccacctctaccctgtctcacggccccccatgccagcctgacggttgtgtctgcctcagtcatgctccatttttccatcgggaccatcaagagggtgtttgtgtctaaggctgactgggtaactttggatgagcggtctctccgctctgagcctgtttcctcatctgtcaaatgggctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 21 - DNA, 544 bp, Artificial Sequence
  Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactg

SEQ ID NO: 22 - DNA, 787 bp, Artificial Sequence
  Synthetic polynucleotide
gtacctcgcgaatgcatctacgatgcgagaacttgtgcctccccgtgttcctgctctttgtccctctgtcctacttagactaatatttgccttgggtactgcaaacaggaaatgggggagggattcgaaacggctttgcggacactagcgatgcgagaacttgtgcctccccgtgttcctgctctttgtccctctgtcctacttagactaatatttgccttgggtactgcaaacaggaaatgggggagggattcgaaacggctttgcggacactagtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaac